(19) United States
(12) Patent Application Publication
(10) Pub. No.: US 2004/0242897 A1
(43) Pub. Date: Dec. 2, 2004

(54) Universal support media for synthesis of oligomeric compounds

(76) Inventors: Andrei P. Guzaev, Vista, CA (US); Muthiah Manoharan, Weston, MA (US)

Correspondence Address: WOODCOCK WASHBURN LLP, ONE LIBERTY PLACE - 46TH FLOOR, PHILADELPHIA, PA, 19103, US

(21) Appl. No.: 10/733,831

(22) Filed: Dec. 10, 2003

Related U.S. Application Data
(63) Continuation-in-part of application No. PCT/US03/23344, filed on Jul. 25, 2003.
Application No. PCT/US03/23344 is a continuation-in-part of application No. 10/260,076, filed on Sep. 30, 2002, now Pat. No. 6,653,468.
(60) Provisional application No. 60/400,312, filed on Jul. 31, 2002.

(51) Int. Cl. G01N 33/53; C07D 333/78; C07D 491/02; C07D 495/02

(52) U.S. Cl. 548/453; 549/49; 549/459; 435/7.1

(57) ABSTRACT

Compounds for the synthesis of oligomeric compounds, particularly oligonucleotides and oligonucleotide mimetics, are provided. In addition, methods for functionalizing a support medium with a first monomeric subunit and methods for the synthesis of oligomeric compounds utilizing the novel compounds bound to support media are provided.

### CROSS REFERENCE TO RELATED APPLICATIONS

This application is a continuation-in-part application of PCT/US03/2334 filed Jul., 25, 2003, which is a continuation-in-part application of U.S. patent application Ser. No. 10/260,076 filed Sep. 30, 2002 now issued U.S. Pat. No. 6,653,468 issued Nov. 25, 2003, which claims benefit of U.S. Provisional Application Serial No. 60/400,312 filed Jul. 31, 2002, the entirety of each application is herein incorporated by reference.

### FIELD OF THE INVENTION

This invention is directed in one aspect to compounds useful in the preparation of novel universal support media. The universal support media thus prepared are useful in the preparation of oligomeric compounds.

### BACKGROUND OF THE INVENTION

Support bound oligonucleotide synthesis relies on sequential addition of nucleotides to one end of a growing chain. Typically, a first nucleoside is attached to an appropriate support medium such as a glass bead support and activated phosphorus compounds (typically nucleotide phosphoramidites, also bearing appropriate protecting groups) are added stepwise to elongate the growing oligonucleotide. When the chain elongation is completed, the oligonucleotide is cleaved from its support and protecting groups are removed. Additional methods for support bound synthesis methods may be found in Caruthers U.S. Pat. Nos. 4,415,732; 4,458,066; 4,500,707; 4,668,777; 4,973,679; and 5,132,418; and Koster U.S. Pat. Nos. 4,725,677 and Re. 34,069.

In carrying out standard oligonucleotide syntheses, workers minimally need to maintain a supply of eight different nucleoside-loaded supports for DNA and RNA syntheses, each prederivatized with a separate nucleoside corresponding to the 3′ terminus of the desired oligomer (adenosine, guanosine, cytidine, uridine, deoxyadenosine, deoxyguanosine, deoxycytidine, and thymidine). If a modified nucleoside is desired at the 3′-terminus then additional prederivatized supports are required. Typically, the first nucleoside is covalently bound by a succinate or hydroquinone-O,O′-diacetate linker. Furthermore, certain oligonucleotides with unusual nucleosides are available only as phosphoroamidites but not as supports.

A universal support is a support that may be used as a starting point for oligonucleotide synthesis regardless of the nucleoside species at the 3′ end of the sequence. A universal support has broad application and remedies the aforementioned deficiencies of standard oligonucleotide synthesis procedures because only one support is needed to carry out the oligonucleotide synthesis regardless of what base is desired at the 3′end. This simplifies the synthetic strategy, reduces the number of required reagents in inventory and reduces the likelihood of errors in parallel synthesis applications.

Some researchers have employed derivatized glass supports with 2′(3′)-O-benzyoluridine 5′-O-succinyl so that the uridine moiety is linked to the glass via a succinate linkage [deBear et al., Nucleosides and Nucleotides 6, 821-830 (1987)]. Oligonucleotide synthesis takes place by adding nucleotide monomers to the 2′ or 3′ position of the uridine. Following the synthesis, the newly synthesized oligonucleotide is released from the glass, deprotected and cleaved from the uridinyl terminus in one reaction. Since it is cleaved from the solid support in the cleaving reaction, the uridinyl functionality is no longer available for subsequent oligonucleotide syntheses.

In a similar approach, Crea et al. prepared the dimer 5′-O-p-chlorophenylphospho-2′(3′)-O-acetyluridinyl-[2′-(3′)-3′]-5′-O-dimethoxytritylthymidine p-chlorophenylester and attached the dimer to cellulose via a phosphate linkage. The 5′ position of the thymidine is available for oligonucleotide attachment and synthesis. [Crea et al., Nucleic Acids Research 8, 2331 (1980)]. Aqueous concentrated ammonia is used to the release of the synthesized oligonucleotide from the cellulose leaving the uridine portion of the dimer attached to the cellulose. Although Crea et al. utilized the reactive vicinal groups on the uridine as the release site for the oligonucleotide from the uridine the solid support suggested in this reference is not truly a universal solid support because the 3′-terminal oligonucleotide is incorporated in the solid support reagent and a different support is required for oligonucleotides incorporating a different first nucleoside.

Schwartz et al. attached an adapter, 2′-(3′)-O-dimethoxytrityl-3′-(2′)-O-benzoyluridine-5′-O-(2-cyanoethyl-N,N-diisopropylphosphoramidite, to a thymidine derivatized polystyrene and synthesized an oligonucleotide from the O-dimethoxytrityl position of the uridine [Schwartz et al., Tetrahedron Letters, 36, 1, 27-30, 1995]. While this approach provides a universal solid support for oligonucleotide synthesis, cleavage releases both the adapter and the thymidine from the support and then the synthesized oligonucleotide from the uridine. Thus, thymidine linker must be removed as an impurity and the solid support is unavailable for subsequent reactions.

Some universal supports require cleavage under conditions supplemental to ammonium hydroxide, [Lyttle et al., Nucleic Acids Research, 1996, 24, 14, 2793-2798] making them less useful in many conventional syntheses where ammonium hydroxide is used as cleavage reagent.

The compounds, compositions and processes of the invention provide novel universal support media useful for preparing oligomeric compounds, including oligonucleotides and oligonucleotide mimetics, which may be effectively cleaved without rendering the support media unavailable for subsequent reactions.

### BRIEF SUMMARY OF THE INVENTION

In one embodiment, the invention is directed to compounds of Formula I:

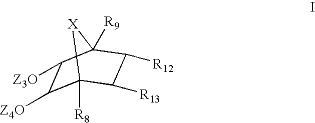

wherein:

X is CR₁₀R₁₁, O, S or NR₃;

R₃ is C₁-C₁₀ alkyl, substituted C₁-C₁₀ alkyl, —C(═O)alkyl, aryl or an amino protecting group;

each R₁₀ and R₁₁ is, independently, H, C₁-C₁₀ alkyl or substituted C₁-C₁₀ alkyl;

each R₁₂ and R₁₃ is, independently, H, C₁-C₁₀ alkyl, substituted C₁-C₁₀ alkyl, —C(═O)—R₄ or —C(═S)—R₄;

R₄ is —O—C₁-C₁₀ alkyl, —O—C₁-C₁₀ substituted alkyl, —O-aryl or —N(J₁)J₂;

J₁ is H or alkyl;

J₂ is alkyl or a nitrogen protecting group;

or J₁ and J₂ together with the nitrogen atom to which they are attached form a ring structure;

each R₈ and R₉ is, independently, H, C₁-C₁₀ alkyl or substituted C₁-C₁₀ alkyl;

each alkyl substituent is, independently, protected hydroxyl, alkoxy, benzyl, nitro, thioalkyl, aryl, thioaryl, thio substituted aryl, thioalkoxy, or halo;

one of Z₃ and Z₄ is a H or a hydroxyl protecting group and the other of Z₃ and Z₄ is a hydroxyl protecting group or -(L)ₙ-sm wherein when both Z₃ and Z₄ are hydroxyl protecting groups said protecting groups are orthogonal to each other; and

L is a linking moiety;

n is 0 or 1; and

sm is a support medium.

In one embodiment of the present invention X is O, CH₂, S or NR₃ with O or CH₂ being preferred. In another embodiment X is O, one of Z₃ and Z₄ is -(L)ₙ-sm and the other of Z₃ and Z₄ a hydroxyl protecting group or H. In a further embodiment X is NR₃ and R₃ is alkyl or —C(═O)alkyl.

In one preferred embodiment R₈ and R₉ are both H. In a further embodiment L is succinyl, oxalyl, —C(═O)— or —C(═O)—NH—.

In one embodiment R₁₂ and R₁₃ is H and the other of R₁₂ and R₁₃ is C₁-C₁₀ alkyl or substituted C₁-C₁₀ alkyl. one of R₁ and R₂ is —N(H)alkyl or N-piperidinyl. In another embodiment R₁₂ and R₁₃ are each H. In a further embodiment one of Z₃ and Z₄ is -(L)ₙ-sm and the other of Z₃ and Z₄ is a hydroxyl protecting group where preferred linking groups L are succinyl or oxalyl groups and a preferred hydroxyl protecting group is dimethoxytrityl.

In one embodiment one of Z₃ and Z₄ is trimethylsilyl, triethylsilyl, t-butyldimethyl-silyl, t-butyldiphenylsilyl, triphenylsilyl, benzoylformyl, acetyl, chloroacetyl, dichloroacetyl, trichloroacetyl, trifluoroacetyl, pivaloyl, benzoyl, p-phenylbenzoyl, 9-fluorenylmethoxy-carbonyl, levulinyl or acetoacetyl groups, and the other of of Z₃ and Z₄ is 4,4′-dimethoxy-trityl, monomethoxytrityl, 9-phenylxanthen-9-yl, 9-(p-methoxyphenyl)xanthen-9-yl, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p,p-dinitrobenzhydryl, p-nitrobenzyl, triphenylmethyl, trimethylsilyl, triethylsilyl, t-butyldimethylsilyl, t-butyldiphenylsilyl, triphenylsilyl, benzoylformate, acetyl, chloroacetyl, trichloroacetyl, trifluoroacetyl, pivaloyl, benzoyl, p-phenylbenzoyl, mesyl, tosyl, 4,4′,4″-tris-(benzyloxy)trityl, 4,4′,4″-tris-(4,5-dichlorophthalimido)trityl, 4,4′,4″-tris(levulinyloxy)trityl, 3-(imidazolylmethyl)-4,4′-dimethoxytrityl, 4-decyloxytrityl, 4-hexadecyloxytrityl, 9-(4-octadecyloxyphenyl)xanthene-9-yl, 1,1-bis-(4-methoxyphenyl)-1′-pyrenyl methyl, p-phenylazophenyloxycarbonyl, 9-fluorenylmethoxycarbonyl, 2,4-dinitrophenylethoxycarbonyl, 4-(methylthiomethoxy)butyryl, 2-(methylthiomethoxymethyl)-benzoyl, 2-(isopropylthiomethoxymethyl)benzoyl, 2-(2,4-dinitrobenzenesulphenyloxymethyl)benzoyl, or a levulinyl group.

In one embodiment of the present invention one of Z₃ and Z₄ has the formula:

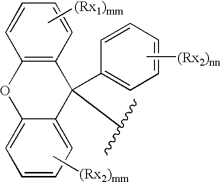

wherein

each Rx₁ is, independently, C₁-C₁₀ alkyl or branched C₁-C₁₀ alkyl;

each Rx₂ is, independently, C₁-C₁₀ alkyl or branched C₁-C₁₀ alkyl;

each Rx₃ is, independently, C₁-C₁₀ alkyl or branched C₁-C₁₀ alkyl;

each mm is, independently, 0, 1, 2 or 3; and

nn is 0, 1, 2 or 3.

In one embodiment nn is 0 and in another embodiment nn is 1 and each Rx₃ is C₁-C₁₀ alkyl. In another embodiment Rx₃ is methyl in the para or ortho position of the phenyl ring. In a further embodiment the other of Z₃ and Z₄ is H.

In a preferred embodiment one of Z₃ and Z₄ has one of the formulas:

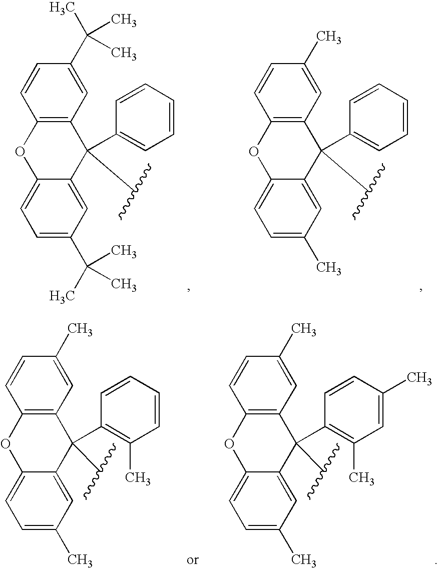

In a preferred embodiment the other of Z₃ and Z₄ is H.

In a preferred embodiment the compounds have formula III:

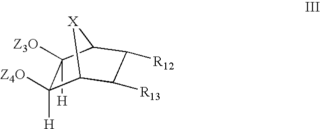

wherein:

X is O or CH₂;

each R₁₂ and R₁₃ is, independently, H, C₁-C₁₀ alkyl, substituted C₁-C₁₀ alkyl, —C(═O)—R₄ or —C(═S)—R₄;

R₄ is —O—C₁-C₁₀ alkyl, —O—C₁-C₁₀ substituted alkyl, —O-aryl or —N(J₁)J₂;

J₁ is H or alkyl;

J₂ is alkyl or a nitrogen protecting group;

or J₁ and J₂ together with the nitrogen atom to which they are attached form a ring structure;

one of Z₃ and Z₄ is a H or a hydroxyl protecting group and the other of Z₃ and Z₄ is a hydroxyl protecting group or -(L)ₙ-sm wherein when both Z₃ and Z₄ are hydroxyl protecting groups said protecting groups are orthogonal to each other;

L is a linking moiety;

n is 0 or 1; and

sm is a support medium.

In a preferred embodiment one of Z₃ and Z₄ is -(L)ₙ-sm and the other of Z₃ and Z₄ a hydroxyl protecting group or H where succinyl and oxalyl groups are preferred for L. A preferred group for the other of Z₃ and Z₄ is dimethoxytrityl.

In one embodiment the support medium is a controlled pore glass, oxalyl-controlled pore glass, silica-containing particles, polymers of polystyrene, copolymers of polystyrene, copolymers of styrene and divinylbenzene, copolymers of dimethylacrylamide and N,N′-bisacryloylethylenediamine, soluble support medium, or PEPS.

In one embodiment preferred support media includes controlled pore glass, polymers of polystyrene or copolymers of polystyrene.

The present invention also provides methods for functionalizing a support medium with a first monomeric subunit, comprising:

providing a support bound compound of Formula II:

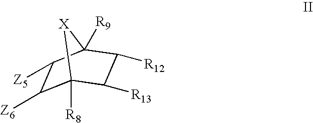

wherein:

X is CR₁₀R₁₁, O, S or NR₃;

R₃ is C₁-C₁₀ alkyl, substituted C₁-C₁₀ alkyl, —C(═O)alkyl, aryl or an amino protecting group;

each R₁₀ and R₁₁ is, independently, H, C₁-C₁₀ alkyl or substituted C₁-C₁₀ alkyl;

each R₁₂ and R₁₃ is, independently, H, C₁-C₁₀ alkyl, substituted C₁-C₁₀ alkyl, —C(═O)—R₄ or —C(═S)—R₄;

R₄ is —O—C₁-C₁₀ alkyl, —O—C₁-C₁₀ substituted alkyl, —O-aryl or —N(J₁)J₂;

J₁ is H or alkyl;

J₂ is alkyl or a nitrogen protecting group;

or J₁ and J₂ together with the nitrogen atom to which they are attached form a ring structure;

each R₈ and R₉ is, independently, H, C₁-C₁₀ alkyl or substituted C₁-C₁₀ alkyl;

each alkyl substituent is, independently, protected hydroxyl, alkoxy, benzyl, nitro, thioalkyl, aryl, thioaryl, thio substituted aryl, thioalkoxy, or halo;

one of Z₅ and Z₆ is a protected hydroxyl group and the other of Z₅ and Z₆ is —O-(L)ₙ-sm;

L is a linking moiety;

n is 0 or 1; and

sm is a support medium;

deprotecting said protected hydroxyl group to give a reactive hydroxyl group; and

treating said reactive hydroxyl group with a first monomeric subunit having an activated phosphorus group and a further protected hydroxyl group thereon for a time and under conditions sufficient to form a monomer-functionalized support medium.

The method optionally further comprises treating the monomer-functionalized support medium with a capping agent and optionally, treating the monomer-functionalized support medium with an oxidizing agent.

The method optionally further comprises deblocking the further protected hydroxyl group to give a reactive hydroxyl group;

treating the reactive hydroxyl group with a further monomeric subunit having an activated phosphorus group and a further protected hydroxyl group thereon for a time and under conditions sufficient to form an extended compound;

treating the extended compound with a capping agent;

optionally, treating said extended compound with an oxidizing or sulfurizing agent;

repeating the preceding four steps one or more times to form a further extended compound; and

treating the further extended compound with an oxidizing or sulfurizing agent to form an oligomeric compound.

In one embodiment the treatment of the further extended compound with the oxidizing agent to form the oligomeric compound removes protecting groups present on the oligomeric compound.

In one embodiment the oligomeric compound is further treated with a reagent effective to cleave the oligomeric compound from the support medium wherein a preferred cleaving agent is a solution of ammonia.

In a preferred embodiment the cleaved oligomeric compound has a terminal hydroxyl group at the site of cleavage which is preferably at the 2′- or 3′-position of the nucleoside that is located at the 3′-terminus of the oligomeric compound. In a preferred embodiment the terminal hydroxyl group is attached to the 2′-position of the nucleoside that is located at the 3′-terminus of the oligomeric compound.

In one embodiment the method includes treating the reactive hydroxyl group with a further monomeric subunit is performed in the presence of an activating agent.

In one embodiment one of Z₃ and Z₄ is -(L)ₙ-sm and the other of Z₃ and Z₄ a hydroxyl protecting group or H where preferred linking groups L are succinyl, oxalyl, —C(═O)— or —C(═O)—NH—.

In a preferred embodiment the method utilizes support medium that include controlled pore glass, oxalyl-controlled pore glass, silica-containing particles, polymers of polystyrene, copolymers of polystyrene, copolymers of styrene and divinylbenzene, copolymers of dimethylacrylamide and N,N′-bisacryloylethylenediamine, soluble support medium and PEPS. Preferred support medium include controlled pore glass, polymers of polystyrene or copolymers of polystyrene.

In one embodiment the compounds used in the method have one of Z₃ and Z₄ being trimethylsilyl, triethylsilyl, t-butyldimethylsilyl, t-butyldiphenylsilyl, triphenylsilyl, benzoylformyl, acetyl, chloroacetyl, dichloroacetyl, trichloroacetyl, trifluoroacetyl, pivaloyl, benzoyl, p-phenylbenzoyl, 9-fluorenylmethoxycarbonyl, levulinyl or acetoacetyl and the other of of Z₃ and Z₄ is 4,4′-dimethoxytrityl, monomethoxytrityl, 9-phenylxanthen-9-yl, 9-(p-methoxyphenyl) xanthen-9-yl, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p,p-dinitrobenzhydryl, p-nitrobenzyl, triphenylmethyl, trimethylsilyl, triethylsilyl, t-butyldimethylsilyl, t-butyldiphenylsilyl, triphenylsilyl, benzoylformate, acetyl, chloroacetyl, trichloroacetyl, trifluoroacetyl, pivaloyl, benzoyl, p-phenylbenzoyl, mesyl, tosyl, 4,4′,4″-tris-(benzyloxy)trityl, 4,4′,4″-tris-(4,5-dichlorophthalimido)trityl, 4,4′,4″-tris(levulinyloxy)trityl, 3-(imidazolylmethyl)-4,4′-dimethoxytrityl, 4-decyloxytrityl, 4-hexadecyloxytrityl, 9-(4-octadecyloxyphenyl)xanthene-9-yl, 1,1-bis-(4-methoxyphenyl)-1′-pyrenyl methyl, p-phenylazophenyloxycarbonyl, 9-fluorenylmethoxycarbonyl, 2,4-dinitrophenylethoxy carbonyl, 4-(methylthiomethoxy)butyryl, 2-(methylthiomethoxymethyl)-benzoyl, 2-(isopropylthiomethoxymethyl)benzoyl, 2-(2,4-dinitrobenzenesulphenyloxymethyl)benzoyl, or a levulinyl group.

In one embodiment the monomeric subunit having an activated phosphorus group is a phosphoramidite, an H-phosphonate or a phosphate triester where phosphoramidite is preferred.

In one embodiment Z₅ is an acid labile hydroxyl-protecting group. In another embodiment the further hydroxyl protecting group is acid labile.

In one embodiment the further hydroxyl protecting group are removed by contact with an acid, wherein the acid is formic acid, acetic acid, chloroacetic acid, dichloroacetic acid, trichloroacetic acid, trifluoroacetic acid, benzenesulfonic acid, toluenesulfonic acid, or phenylphosphoric acid.

In one embodiment the oligomeric compound is an oligonucleotide, modified oligonucleotide, oligonucleotide analog, oligonucleoside, oligonucleotide mimetic, hemimer, gapmer or chimera. A preferred oligomeric compound is an oligonucleotide.

In a preferred embodiment one of Z₅ and Z₆ has the formula:

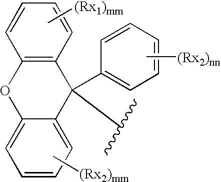

wherein

each Rx₁ is, independently, C₁-C₁₀ alkyl or branched C₁-C₁₀ alkyl;

each Rx₂ is, independently, C₁-C₁₀ alkyl or branched C₁-C₁₀ alkyl;

each Rx₃ is, independently, C₁-C₁₀ alkyl or branched C₁-C₁₀ alkyl;

each mm is, independently, 0, 1, 2 or 3; and

nn is 0, 1, 2 or 3.

In one embodiment nn is 0. In another embodiment nn is 1 and Rx₃ is C₁-C₁₀ alkyl. In a further embodiment Rx₃ is methyl in the para or ortho position of the phenyl ring.

### DETAILED DESCRIPTION OF THE INVENTION

The present invention provides compounds and processes useful for the support mediated synthesis of oligomeric compounds. Compounds of the invention are initially attached to support media and subsequently deblocked thereby providing a free hydroxyl group. This free hydroxyl group is used for oligomer synthesis in an analogous manner to the free 5′-hydroxyl group that is provided when using a nucleoside derivatized commercially supplied support medium. In one embodiment, the free hydroxyl group of the universal support medium may be reacted with a monomeric subunit having an activated phosphorus group to form a phosphite linkage. The synthesis continues in this manner iteratively until the desired oligomeric compound is prepared. The traditional iterative steps include oxidation, capping and deblocking. When the desired sequence has been iteratively synthesized, the oligomeric compound is cleaved from the support media. The oligomeric comound thus synthesized will have a terminal hydroxyl group and not incorporate the compound that supplied the initial free hydroxyl group to initiate synthesis. The synthesis is similar to conventional oligomeric compound synthesis but the compounds of the present invention are used to prepare support media that can be used to prepare any sequence. This eliminates having a support medium that has been prepared having a specific monomeric subunit attached that will be the terminal subunit upon cleavage.

As used herein, the term “orthogonally protecting groups” refers to functional groups that are protected with different classes of protecting groups, wherein each class of protecting group can be removed in any order and in the presence of all other classes (see, Barany, G. and Merrifield, R. B., J. Am. Chem. Soc., 1977, 99, 7363; idem, 1980, 102, 3084.) Orthogonal protection is widely used in, for example, automated oligonucleotide synthesis. A functional group is deblocked in the presence of one or more other protected functional groups that is not affected by the deblocking procedure. This deblocked functional group is reacted in some manner and at some point a further orthogonal protecting group is removed under a different set of reaction conditions. This allows for selective chemistry to arrive at a desired compound or oligomeric compound.

In the context of this invention, the term “oligomeric compound” refers to a polymeric structure capable of being prepared using well-known support mediated synthetic methods. Preferred oligomeric compounds are also capable of hybridizing a region of a nucleic acid molecule. The term includes oligonucleotides, oligonucleosides, oligonucleotide analogs, modified oligonucleotides, oligonucleotide mimetics, hemimers, gapmers and chimeras. Oligomeric compounds can be prepared to be linear or circular and may include branching. They can be prepared single stranded or double stranded and may include overhangs. In general, an oligomeric compound comprises a backbone of linked momeric subunits where each linked momeric subunit is directly or indirectly attached to a heterocyclic base moiety. The linkages joining the monomeric subunits, the monomeric subunits and the heterocyclic base moieties can be variable in structure giving rise to a plurality of motifs for the resulting oligomeric compounds, including hemimers, gapmers and chimeras.

As is known in the art, a nucleoside is a base-sugar combination. The base portion of the nucleoside is normally a heterocyclic base moiety. The two most common classes of such heterocyclic bases are purines and pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. The respective ends of this linear polymeric structure can be joined to form a circular structure by hybridization or by formation of a covalent bond, however, open linear structures are generally preferred. Within the oligonucleotide structure, the phosphate groups are commonly referred to as forming the intemucleoside linkages of the oligonucleotide. The normal intemucleoside linkage of RNA and DNA is a 3′ to 5′ phosphodiester linkage.

In the context of this invention, the term “oligonucleotide” refers to an oligomer or polymer of ribonucleic acid (RNA) or deoxyribonucleic acid (DNA). This term includes oligonucleotides composed of naturally-occurring nucleobases, sugars and covalent internucleoside linkages. The terms “oligonucleotide analog” and “modified oligonucleotide” refers to oligonucleotides that have one or more non-naturally occurring portions which function in a similar manner to oligonulceotides. Such modified or substituted oligonucleotides are often preferred over native forms because of desirable properties such as, for example, enhanced cellular uptake, enhanced affinity for nucleic acid target and increased stability in the presence of nucleases.

In the context of this invention, the term “oligonucleoside” refers to nucleosides that are joined by intemucleoside linkages that do not have phosphorus atoms. Intemucleoside linkages of this type include short chain alkyl, cycloalkyl, mixed heteroatom alkyl, mixed heteroatom cycloalkyl, one or more short chain heteroatomic and one or more short chain heterocyclic. These intemucleoside linkages include but are not limited to siloxane, sulfide, sulfoxide, sulfone, acetyl, formacetyl, thioformacetyl, methylene formacetyl, thioformacetyl, alkeneyl, sulfamate; methyleneimino, methylenehydrazino, sulfonate, sulfonamide, amide and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

In the context of this invention, the term “oligonucleotide mimetic” refers to an oligonucleotide wherein the backbone of the nucleotide units has been replaced with novel groups. Although the term is intended to include oligomeric compounds wherein only the furanose ring or both the furanose ring and the internucleotide linkage are replaced with novel groups, replacement of only the furanose ring is also referred to in the art as being a sugar surrogate. Oligonucleotide mimetics can be further modified to incorporate one or more modified heterocyclic base moieties to enhance properties such as hybridization.

One class of oligonucleotide mimetic that has been reported to have excellent hybridization properties is peptide nucleic acids (PNA). The backbone in PNA compounds is two or more linked aminoethylglycine units that give PNA an amide containing backbone. The heterocyclic base moieties are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

PNA has been modified to incorporate numerous modifications since the basic PNA structure was first prepared. The basic structure is shown below:

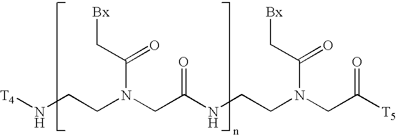

wherein

Bx is a heterocyclic base moiety;

T₄ is is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are hydroxyl, amino, alkoxy, carboxy, benzyl, phenyl, nitro, thiol, thioalkoxy, halogen, alkyl, aryl, alkenyl or alkynyl;

T₅ is —OH, —N(Z₁)Z₂, R₅, D or L α-amino acid linked via the α-amino group or optionally through the ω-amino group when the amino acid is lysine or omithine or a peptide derived from D, L or mixed D and L amino acids linked through an amino group, a chemical functional group, a reporter group or a conjugate group;

Z₁ is hydrogen, C₁-C₆ alkyl, or an amino protecting group;

Z₂ is hydrogen, C₁-C₆ alkyl, an amino protecting group, —C(═O)—(CH₂)ₙ-J-Z₃, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group;

Z₃ is hydrogen, an amino protecting group, —C₁-C₆ alkyl, —C(═O)—CH₃, benzyl, benzoyl, or —(CH₂)ₙ—N(H)Z₁;

each J is O, S or NH;

R₅ is a carbonyl protecting group; and

n is from 2 to about 50.

Another class of oligonucleotide mimetic that has been studied is based on linked morpholino units (morpholino nucleic acid) having heterocyclic bases attached to the morpholino ring. A number of intermonomer linking groups have been reported that link the morpholino monomeric units in a morpholino nucleic acid. A preferred class of linking groups has been selected to give a non-ionic oligomeric compound. The non-ionic morpholino-based oligomeric compounds are less likely to have undesired interactions with cellular proteins. Morpholino-based oligomeric compounds are non-ionic mimics of oligonucleotides that are less likely to form undesired interactions with cellular proteins (Dwaine A. Braasch and David R. Corey, Biochemistry, 2002, 41(14), 4503-4510). Morpholino-based oligomeric compounds are disclosed in U.S. Pat. No. 5,034,506, issued Jul. 23, 1991. The morpholino class of oligomeric compounds has been prepared having a variety of different linking groups joining the monomeric subunits.

Morpholino nucleic acids have been prepared having a variety of different linking groups (L₂) joining the monomeric subunits. The basic formula is shown below:

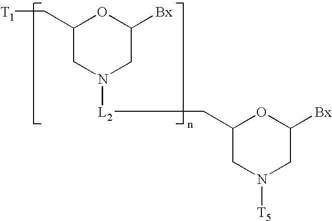

wherein:

T₁ is hydroxyl or a protected hydroxyl;

T₅ is hydrogen or a phosphate or phosphate derivative;

L₂ is a linking group; and

n is from 2 to about 50.

A further class of oligonucleotide mimetic is referred to as cyclohexenyl nucleic acids (CeNA). The furanose ring normally present in an DNA/RNA molecule is replaced with a cyclohenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general the the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation. Furthermore the incorporation of CeNA into a sequence targeting RNA was stable to serum and able to activate E. Coli RNase resulting in cleavage of the target RNA strand.

The general formula of CeNA is shown below:

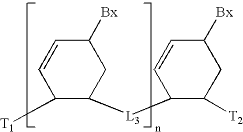

wherein:

each Bx is a heterocyclic base moiety;

T₁ is hydroxyl or a protected hydroxyl; and

T2 is hydroxyl or a protected hydroxyl.

Another class of oligonucleotide mimetic (anhydrohexitol nucleic acid) can be prepared from one or more anhydrohexitol nucleosides (see, Wouters and Herdewijn, Bioorg. Med. Chem. Lett., 1999, 9, 1563-1566) and would have the general formula:

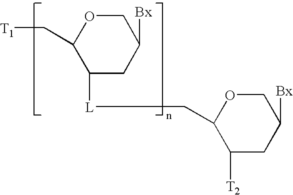

A further prefered modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage is preferably a methelyne (—CH2-)n group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10 C), stability towards 3′-exonucleolytic degradation and good solubility properties. The basic structure of LNA showing the bicyclic ring system is shown below:

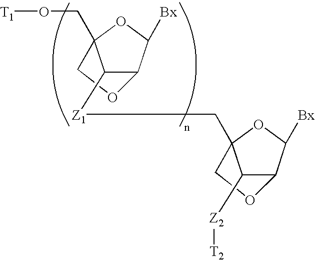

The conformations of LNAs determined by 2D NMR spectroscopy have shown that the locked orientation of the LNA nucleotides, both in single-stranded LNA and in duplexes, constrains the phosphate backbone in such a way as to introduce a higher population of the N-type conformation (Petersen et al., J. Mol. Recognit., 2000, 13, 44-53). These conformations are associated with improved stacking of the nucleobases (Wengel et al., Nucleosides Nucleotides, 1999, 18, 1365-1370).

LNA has been shown to form exceedingly stable LNA:LNA duplexes (Koshkin et al., J. Am. Chem. Soc., 1998, 120, 13252-13253). LNA:LNA hybridization was shown to be the most thermally stable nucleic acid type duplex system, and the RNA-mimicking character of LNA was established at the duplex level. Introduction of 3 LNA monomers (T or A) significantly increased melting points (Tm=+15/+11) toward DNA complements. The universality of LNA-mediated hybridization has been stressed by the formation of exceedingly stable LNA:LNA duplexes. The RNA-mimicking of LNA was reflected with regard to the N-type conformational restriction of the monomers and to the secondary structure of the LNA:RNA duplex.

LNAs also form duplexes with complementary DNA, RNA or LNA with high thermal affinities. Circular dichroism (CD) spectra show that duplexes involving fully modified LNA (especially LNA:RNA) structurally resemble an A-form RNA:RNA duplex. Nuclear magnetic resonance (NMR) examination of an LNA:DNA duplex confirmed the 3′-endo conformation of an LNA monomer. Recognition of double-stranded DNA has also been demonstrated suggesting strand invasion by LNA. Studies of mismatched sequences show that LNAs obey the Watson-Crick base pairing rules with generally improved selectivity compared to the corresponding unmodified reference strands.

Novel types of LNA-modified oligonucleotides, as well as the LNAs, are useful in a wide range of diagnostic and therapeutic applications. Among these are antisense applications, PCR applications, strand-displacement oligomers, substrates for nucleic acid polymerases and generally as nucleotide-based drugs.

Potent and nontoxic antisense oligonucleotides containing LNAs have been described (Wahlestedt et al., Proc. Natl. Acad. Sci. U.S.A., 2000, 97, 5633-5638.) The authors have demonstrated that LNAs confer several desired properties to antisense agents. LNA/DNA copolymers were not degraded readily in blood serum and cell extracts. LNA/DNA copolymers exhibited potent antisense activity in assay systems as disparate as G-protein-coupled receptor signaling in living rat brain and detection of reporter genes in Escherichia coli. Lipofectin-mediated efficient delivery of LNA into living human breast cancer cells has also been accomplished.

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

The first analogs of LNA, phosphorothioate-LNA and 2′-thio-LNAs, have also been prepared (Kumar et al., Bioorg. Med. Chem. Lett., 1998, 8, 2219-2222). Preparation of locked nucleoside analogs containing oligodeoxyribonucleotide duplexes as substrates for nucleic acid polymerases has also been described (Wengel et al, PCT International Application WO 98-DK393 19980914). Furthermore, synthesis of 2′-amino-LNA, a novel conformationally restricted high-affinity oligonucleotide analog with a handle has been described in the art (Singh et al., J. Org. Chem., 1998, 63, 10035-10039). In addition, 2′-amino- and 2′-methylamino-LNAs have been prepared and the thermal stability of their duplexes with complementary RNA and DNA strands has been previously reported.

Further oligonucleotide mimetics have been prepared to incude bicyclic and tricyclic nucleoside analogs having the formulas (amidite monomers shown):

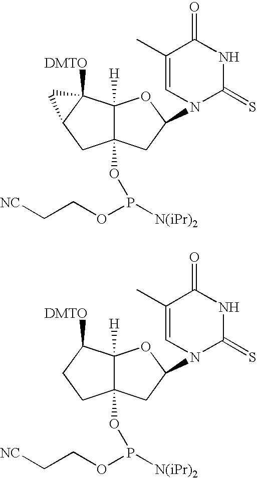

(see Steffens et al., Helv. Chim. Acta, 1997, 80, 2426-2439; Steffens et al., J. Am. Chem. Soc., 1999, 121, 3249-3255; and Renneberg et al., J. Am. Chem. Soc., 2002, 124, 5993-6002). These modified nucleoside analogs have been oligomerized using the phosphoramidite approach and the resulting oligomeric compounds containing tricyclic nucleoside analogs have shown increased thermal stabilities (Tm's) when hybridized to DNA, RNA and itself. Oligomeric compounds containing bicyclic nucleoside analogs have shown thermal stabilities approaching that of DNA duplexes.

Another class of oligonucleotide mimetic is referred to as phosphonomonoester nucleic acids incorporate a phosphorus group in a backbone the backbone. This class of olignucleotide mimetic is reported to have useful physical and biological and pharmacological properties in the areas of inhibiting gene expression (antisense oligonucleotides, ribozymes, sense oligonucleotides and triplex-forming oligonucleotides), as probes for the detection of nucleic acids and as auxiliaries for use in molecular biology.

The general formula (for definitions of Markush variables see: U.S. Pat. Nos. 5,874,553 and 6,127,346 herein incorporated by reference in their entirety) is shown below.

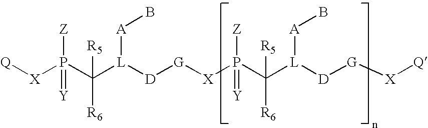

Another oligonucleotide mimetic has been reported wherein the furanosyl ring has been replaced by a cyclobutyl moiety.

The intemucleotide linkage found in native nucleic acids is a phosphodiester linkage. This linkage has not been the linkage of choice for synthetic oligonucleotides that are for the most part targeted to a portion of a nucleic acid such as mRNA because of stability problems e.g. degradation by nucleases. Preferred internucleotide linkages and internucleoside linkages as is the case for non phosphate ester type linkages include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleoside linkages is a 3′ to 3′, 5′ to 5′ or 2′ to 2′ linkage. Preferred oligonucleotides having inverted polarity comprise a single 3′ to 3′ linkage at the 3′-most internucleotide linkage, i.e. a single inverted nucleoside residue that may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

In more preferred embodiments of the invention, oligomeric compounds have one or more phosphorothioate and/or heteroatom intemucleoside linkages, in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester intemucleotide linkage is represented as —O—P(═O)(OH)—O—CH₂—]. The MMI type intemucleoside linkages are disclosed in the above referenced U.S. Pat. No. 5,489,677. Preferred amide intemucleoside linkages are disclosed in the above referenced U.S. Pat. No. 5,602,240.

Oligomeric compounds can have a variety of substituent groups attached at various positions. Furanosyl groups found in native nucleic acids as well as various oligomeric compounds can be substituted at a number of positions. The most frequently substituted position is the 2′-position of ribose. The 3′, 4′, and 5′ have also been substituted with substituent groups generally referred to as sugar substituent groups. Preferred sugar substituent groups include: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other sugar substituent groups include: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties.

More preferred sugar substituent groups that are more frequently covalently attached to the 2′-sugar position include methoxyethoxy (—O—CH₂CH₂OCH₃, also known as —O—(2-methoxyethyl) or MOE) (Martin et al., Helv. Chim. Acta, 1995, 78, 486-504), i.e., an alkoxyalkoxy group. A further preferred 2′-modification includes dimethylaminooxyethoxy, i.e., a —O(CH₂)₂ON(CH₃)₂ group, also known as DMAOE, as described in examples hereinbelow, and -dimethylaminoethoxyethoxy (also known in the art as —O—dimethylaminoethoxyethyl or -DMAEOE), i.e., O—CH₂—O—CH₂—N(CH₂)₂, also described in examples hereinbelow.

Other preferred sugar substituent groups that are more frequently covalently attached to the 2′-sugar position include methoxy (—O—CH₃), aminopropoxy (—OCH₂CH₂CH₂NH₂), allyl (—CH₂—CH═CH₂), —O-allyl (—O—CH₂—CH═CH₂) and fluoro (—F). A 2″-substituent group on a furanosyl ring can be in the ribo (down) or arabino (up) position. Preferred 2′-arabino modifications include fluoro and hydroxy. Similar modifications may also be made at other positions on an oligomeric compound, particularly the 3′ position of the sugar for a 2′-5′ linked oligomeric compound, the 3′-terminus and the 5′-position of the 5′-terminus. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Further representative sugar substituent groups include groups of formula Iₐ or IIₐ:

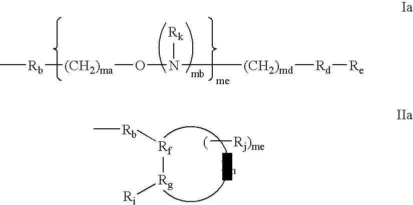

wherein:

R_{b} is O, S or NH;

R_{d} is a single bond, O, S or C(═O);

Rₑ is C₁-C₁₀ alkyl, N(Rₖ)(Rₘ), N(Rₖ)(Rₙ), N═C(Rₚ)(R_{q}), N═C(Rₚ)(Rᵣ) or has formula IIIₐ;

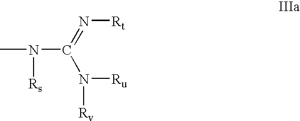

Rₚ and R_{q} are each independently hydrogen or C₁-C₁₀ alkyl;

Rᵣ is —Rₓ—R_{y};

each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;

or optionally, Rᵤ and Rᵥ, together form a phthalimido moiety with the nitrogen atom to which they are attached;

each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;

Rₖ is hydrogen, a nitrogen protecting group or —Rₓ—R_{y};

Rₚ is hydrogen, a nitrogen protecting group or —Rₓ—R_{y};

Rₓ is a bond or a linking moiety;

R_{y} is a chemical functional group, a conjugate group or a solid support medium;

each Rₘ and Rₙ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(Rᵤ)(Rᵥ), guanidino and acyl where said acyl is an acid amide or an ester;

or Rₘ and Rₙ, together, are a nitrogen protecting group, are joined in a ring structure that optionally includes an additional heteroatom selected from N and O or are a chemical functional group;

Rᵢ is OR_{z}, SR_{z}, or N(R_{z})₂;

each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;

R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;

Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ)ORₖ, halo, SRₖ or CN;

mₐ is 1 to about 10;

each mb is, independently, 0 or 1;

mc is 0 or an integer from 1 to 10;

md is an integer from 1 to 10;

me is from 0, 1 or 2; and

provided that when mc is 0, md is greater than 1.

Representative substituents groups of Formula Ia are disclosed in U.S. patent application Ser. No. 09/130,973, filed Aug. 7, 1998, entitled “Capped 2′-Oxyethoxy Oligonucleotides,” hereby incorporated by reference in its entirety.

Representative cyclic substituent groups of Formula IIa are disclosed in U.S. patent application Ser. No. 09/123,108, filed Jul. 27, 1998, entitled “RNA Targeted 2′-Modified Oligonucleotides that are Conformationally Preorganized,” hereby incorporated by reference in its entirety.

Oligomeric compounds may also include nucleobase (often referred to in the art simply as “base” or “heterocyclic base moiety”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

In one aspect of the present invention oligomeric compounds are prepared having polycyclic heterocyclic compounds in place of one or more heterocyclic base moieties. A number of tricyclic heterocyclic comounds have been previously reported. These compounds are routinely used in antisense applications to increase the binding properties of the modified strand to a target strand. The most studied modifications are targeted to guanosines hence they have been termed G-clamps or cytidine analogs. Many of these polycyclic heterocyclic compounds have the general formula:

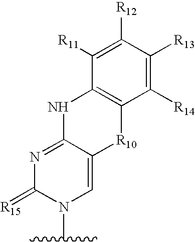

Representative cytosine analogs that make 3 hydrogen bonds with a guanosine in a second strand include 1,3-diazaphenoxazine-2-one (R₁₀=O, R₁₁-R₁₄=H) [Kurchavov, et al., Nucleosides and Nucleotides, 1997, 16, 1837-1846], 1,3-diazaphenothiazine-2-one (R₁₀=S, R₁₁-R₁₄=H), [Lin, K-Y; Jones, R. J.; Matteucci, M. J. Am. Chem. Soc. 1995, 117,3873-3874] and 6,7,8,9-tetrafluoro-1,3-diazaphenoxazine-2-one (R₁₀=O, R₁₁-R₁₄=F) [Wang, J.; Lin, K-Y, Matteucci, M. Tetrahedron Lett. 1998, 39, 8385-8388]. Incorporated into oligonucleotides these base modifications were shown to hybridize with complementary guanine and the latter was also shown to hybridize with adenine and to enhance helical thermal stability by extended stacking interactions (also see U.S. Patent Application entitled “Modified Peptide Nucleic Acids” filed May 24, 2002, Ser. No. 10/155,920; and U.S. Patent Application entitled “Nuclease Resistant Chimeric Oligonucleotides” filed May 24, 2002, Ser. No. 10/013,295, both of which are commonly owned with this application and are herein incorporated by reference in their entirety).

Further helix-stabilizing properties have been observed when a cytosine analog/substitute has an aminoethoxy moiety attached to the rigid 1,3-diazaphenoxazine-2-one scaffold (R₁₀=O, R₁₁=—O—(CH₂)₂—NH₂, R₁₂₋₁₄=H) [Lin, K.-Y.; Matteucci, M., J. Am. Chem. Soc. 1998, 120, 8531-8532]. Binding studies demonstrated that a single incorporation could enhance the binding affinity of a model oligonucleotide to its complementary target DNA or RNA with a ΔTₘ of up to 18° relative to 5-methyl cytosine (dC5^{me}), which is the highest known affinity enhancement for a single modification, yet. On the other hand, the gain in helical stability does not compromise the specificity of the oligonucleotides. The Tₘ data indicate an even greater discrimination between the perfect match and mismatched sequences compared to dC5^{me}. It was suggested that the tethered amino group serves as an additional hydrogen bond donor to interact with the Hoogsteen face, namely the O6, of a complementary guanine thereby forming 4 hydrogen bonds. This means that the increased affinity of G-clamp is mediated by the combination of extended base stacking and additional specific hydrogen bonding.

Further tricyclic heterocyclic compounds and methods of using them that are amenable to the present invention are disclosed in U.S. Pat. No. 6,028,183, which issued on May 22, 2000, and U.S. Pat. No. 6,007,992, which issued on Dec. 28, 1999, the contents of both are commonly assigned with this application and are incorporated herein in their entirety.

The enhanced binding affinity of the phenoxazine derivatives together with their uncompromised sequence specificity makes them valuable nucleobase analogs for the development of more potent antisense-based drugs. In fact, promising data have been derived from in vitro experiments demonstrating that heptanucleotides containing phenoxazine substitutions are capable to activate RNaseH, enhance cellular uptake and exhibit an increased antisense activity [Lin, K-Y; Matteucci, M., J. Am. Chem. Soc. 1998, 120, 8531-8532]. The activity enhancement was even more pronounced in case of G-clamp, as a single substitution was shown to significantly improve the in vitro potency of a 20mer 2′-deoxyphosphorothioate oligonucleotides [Flanagan, W. M.; Wolf, J. J.; Olson, P.; Grant, D.; Lin, K-Y; Wagner, R. W.; Matteucci, M. Proc. Natl. Acad. Sci. USA, 1999, 96, 3513-3518]. Nevertheless, to optimize oligonucleotide design and to better understand the impact of these heterocyclic modifications on the biological activity, it is important to evaluate their effect on the nuclease stability of the oligomers.

Further modified polycyclic heterocyclic compounds useful as heterocyclcic bases are disclosed in but not limited to, the above noted U.S. Pat. No. 3,687,808, as well as U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121, 5,596,091; 5,614,617; 5,645,985; 5,646,269; 5,750,692; 5,830,653; 5,763,588; 6,005,096; and 5,681,941, and U.S. patent application Ser. No. 09/996,292 filed Nov. 28, 2001, certain of which are commonly owned with the instant application, and each of which is herein incorporated by reference.

A further preferred modification of oligomeric compounds of the invention involves the linkage of one or more moieties or conjugates which enhance the activity, cellular distribution or cellular uptake of the resulting oligomeric compounds. In one embodiment such modified oligomeric compounds are prepared by covalently attaching conjugate groups to functional groups such as hydroxyl or amino groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999), which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

It is not necessary for all positions in a given oligomeric compound to be uniformly modified, and in fact more than one of the aforementioned modifications may be incorporated in a single oligomeric compound or even at a single monomeric subunit such as a nucleoside within an oligomeric compound. The present invention also includes oligomeric compounds that are chimeric compounds. “Chimeric” oligomeric compounds or “chimeras,” in the context of this invention, are oligomeric compounds that contain two or more chemically distinct regions, each made up of at least one monomer unit, i.e., a nucleotide in the case of an oligonucleotide compound. These oligomeric compounds typically contain at least one region modified so as to confer increased resistance to nuclease degradation, increased cellular uptake, and/or increased binding affinity for the target nucleic acid. An additional region of the oligomeric compound may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of inhibition of gene expression. Consequently, comparable results can often be obtained with shorter oligomeric compounds when chimeras are used, compared to for example phosphorothioate deoxyoligonucleotides hybridizing to the same target region. Cleavage of the RNA target can be routinely detected by gel electrophoresis and, if necessary, associated nucleic acid hybridization techniques known in the art.

Chimeric oligomeric compounds of the invention may be formed as composite structures of two or more oligonucleotides, modified oligonucleotides, oligonucleotide analogs, oligonucleosides and/or oligonucleotide mimetics as described above. Such compounds have also been referred to in the art as hybrids hemimers, gapmers or inverted gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyl-adenine, 5-propynyl-uracil and 5-propynyl-cytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Further modified nucleobases include tricyclic heterocyclic base moieties such as for example 1,3-diazaphenoxazine-2-one (1H-pyrimido[5,4-b][1,4]benzoxazin-2(3H)-one) and G-clamps such as 9-(2-aminoethoxy)-1,3,-diazaphenoxazine-2-one. Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,750,692; 5,830,653; 5,763,588; 6,005,096; and 5,681,941, certain of which are commonly owned with the instant application, and each of which is herein incorporated by reference.

As is known in the art, a nucleoside is a base-sugar combination. The base portion of the nucleoside is normally a heterocyclic base moiety. The two most common classes of such heterocyclic bases are purines and pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. The respective ends of this linear polymeric structure can be joined to form a circular structure by hybridization or by formation of a covalent bond, however, open linear structures are generally preferred. Within the oligonucleotide structure, the phosphate groups are commonly referred to as forming the intemucleoside linkages of the oligonucleotide. The normal intemucleoside linkage of RNA and DNA is a 3′ to 5′ phosphodiester linkage.

In one aspect the compounds useful in the synthesis of oligomeric compounds have Formula I:

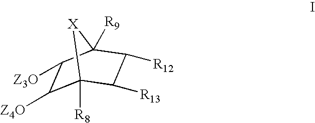

wherein:

X is CR₁₀R₁₁, O, S or NR₃;

R₃ is C₁-C₁₀ alkyl, substituted C₁-C₁₀ alkyl, —C(═O)alkyl, aryl or an amino protecting group;

each R₁₀ and R₁₁ is, independently, H, C₁-C₁₀ alkyl or substituted C₁-C₁₀ alkyl;

each R₁₂ and R₁₃ is, independently, H, C₁-C₁₀ alkyl, substituted C₁-C₁₀ alkyl, —C(═O)—R₄ or —C(═S)—R₄;

R₄ is —O—C₁-C₁₀ alkyl, —O—C₁-C₁₀ substituted alkyl, —O-aryl or —N(J₁)J₂;

J₁ is H or alkyl;

J₂ is alkyl or a nitrogen protecting group;

or J₁ and J₂ together with the nitrogen atom to which they are attached form a ring structure;

each R₈ and R₉ is, independently, H, C₁-C₁₀ alkyl or substituted C₁-C₁₀ alkyl;

each alkyl substituent is, independently, protected hydroxyl, alkoxy, benzyl, nitro, thioalkyl, aryl, thioaryl, thio substituted aryl, thioalkoxy, or halo;

one of Z₅ and Z₆ is a protected hydroxyl group and the other of Z₅ and Z₆ is —O-(L)ₙ-sm;

L is a linking moiety;

n is 0 or 1; and

sm is a support medium.

To prepare compounds of the invention wherein X is S or NR₃, one may begin with starting materials known to those of skill in the art, including the cantharidin analogues disclosed by McCluskey et al., Bioorganic & Medicinal Chemistry Letters (2002), 12(3), 391-393; the bicycloheptenedicarboxylic anhydride derivative polymers disclosed by Besecke et al. in German Application 91-4117369; isobenzofuran-4,7-imine-1,3-dione,3a,4,7,7a-tetrahydro-8-methyl (CA Registry No. 41532-47-2); and; isobenzofuran-4,7-imine-1,3-dione,8-acetyl-3a,4,7,7a-tetrahydro-, (3aα,4α,7α,7aα)-(CA Registry No. 99237-90-8).

Preferably, the support medium is a controlled pore glass, oxalyl-controlled pore glass, silica-containing particles, polymers of polystyrene, copolymers of polystyrene, copolymers of styrene and divinylbenzene, copolymers of dimethylacrylamide and N,N′-bisacryloylethylenediamine, soluble support medium or PEPS.

Preferably, Z₃ is trimethylsilyl, triethylsilyl, t-butyldimethylsilyl, t-butyldiphenylsilyl, triphenylsilyl, benzoylformyl, acetyl, chloroacetyl, dichloroacetyl, trichloroacetyl, trifluoroacetyl, pivaloyl, benzoyl, p-phenylbenzoyl, 9-fluorenylmethoxycarbonyl, levulinyl or acetoacetyl groups.

Preferably, Z₄ is 4,4′-dimethoxytrityl (DMT), monomethoxytrityl, 9-phenylxanthen-9-yl (Pixyl), 9-(p-methoxyphenyl)xanthen-9-yl (Mox), t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p,p-dinitrobenzhydryl, p-nitrobenzyl, triphenylmethyl, trimethylsilyl, triethylsilyl, t-butyldimethylsilyl, t-butyldiphenylsilyl, triphenylsilyl, benzoylformate, acetyl, chloroacetyl, trichloroacetyl, trifluoroacetyl, pivaloyl, benzoyl, p-phenylbenzoyl, mesyl, tosyl, 4,4′,4″-tris-(benzyloxy)trityl (TBTr), 4,4′,4″-tris-(4,5-dichlorophthalimido)trityl (CPTr), 4,4′,4″-tris(levulinyloxy)trityl (TLTr); 3-(imidazolylmethyl)-4,4′-dimethoxytrityl (IDTr), 4-decyloxytrityl (C₁₀Tr), 4-hexadecyloxytrityl (C₁₆Tr), 9-(4-octadecyloxyphenyl)xanthene-9-yl (C₁₈Px), 1,1-bis-(4-methoxyphenyl)-1′-pyrenyl methyl (BMPM), p-phenylazophenyloxycarbonyl (PAPoc), 9-fluorenylmethoxycarbonyl (Fmoc), 2,4-dinitrophenylethoxycarbonyl (DNPEoc), 4-(methylthiomethoxy)butyryl (MTMB), 2-(methylthiomethoxymethyl)-benzoyl (MTMT), 2-(isopropylthiomethoxymethyl)benzoyl (PTMT), 2-(2,4-dinitrobenzenesulphenyloxymethyl)benzoyl (DNBSB), or levulinyl groups.

Other representative hydroxyl protecting groups commonly used in the art may be found in Beaucage, et al., Tetrahedron 1992, 48, 2223; and Greene and Wuts, Protective Groups in Organic Synthesis, Chapter 2, 2d ed, John Wiley & Sons, New York, 1991, each of which are hereby incorporated by reference in their entirety. Preferred protecting groups include dimethoxytrityl (DMT), monomethoxytrityl, 9-phenylxanthen-9-yl (Pixyl) and 9-(p-methoxyphenyl)xanthen-9-yl (Mox).

Chemical functional groups can also be “blocked” by including them in a precursor form. Thus, an azido group can be used considered as a “blocked” form of an amine since the azido group is easily converted to the amine. Further representative protecting groups utilized in oligonucleotide synthesis are discussed in Agrawal, et al., Protocols for Oligonucleotide Conjugates, Eds, Humana Press; New Jersey, 1994; Vol. 26 pp. 1-72.

In one aspect of the present invention the compounds useful in the synthesis of oligomeric compounds have Formula III:

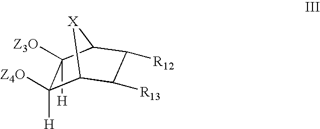

wherein X is O or CH₂;

each R₁₂ and R₁₃ is, independently, H, C₁-C₁₀ alkyl, substituted C₁-C₁₀ alkyl, —C(═O)—R₄ or —C(═S)—R₄;

R₄ is —O—C₁-C₁₀ alkyl, —O—C₁-C₁₀ substituted alkyl, —O-aryl or —N(J₁)J₂;

J₁ is H or alkyl;

J₂ is alkyl or a nitrogen protecting group;

or J₁ and J₂ together with the nitrogen atom to which they are attached form a ring structure;

one of Z₃ and Z₄ is a H or a hydroxyl protecting group and the other of Z₃ and Z₄ is a hydroxyl protecting group or -(L)ₙ-sm wherein when both Z₃ and Z₄ are hydroxyl protecting groups said protecting groups are orthogonal to each other;

L is a linking moiety;

n is 0 or 1; and

sm is a support medium.

In a more preferred embodiment one of Z₃ and Z₄ is a hydroxyl protecting group and the other of Z₃ and Z₄ is an optionally linked support media. Further preferred is O for X and H substitution for both R₁₂ and R₁₃.

The methods of the invention are useful for functionalizing a support medium with a first monomeric subunit. In one embodiment, the method comprises the steps of:

providing a compound of Formula II:

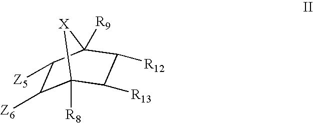

wherein:

X is CR₁₀R₁₁, O, S or NR₃;

R₃ is C₁-C₁₀ alkyl, substituted C₁-C₁₀ alkyl, —C(═O)alkyl, aryl or an amino protecting group;

each R₁₀ and R₁₁ is, independently, H, C₁-C₁₀ alkyl or substituted C₁-C₁₀ alkyl;

each R₁₂ and R₁₃ is, independently, H, C₁-C₁₀ alkyl, substituted C₁-C₁₀ alkyl, —C(═O)—R₄ or —C(═S)—R₄;

R₄ is —O—C₁-C₁₀ alkyl, —O—C₁-C₁₀ substituted alkyl, —O-aryl or —N(J₁)J₂;

J₁ is H or alkyl;

J₂ is alkyl or a nitrogen protecting group;

or J₁ and J₂ together with the nitrogen atom to which they are attached form a ring structure;

each R₈ and R₉ is, independently, H, C₁-C₁₀ alkyl or substituted C₁-C₁₀ alkyl;

each alkyl substituent is, independently, protected hydroxyl, alkoxy, benzyl, nitro, thioalkyl, aryl, thioaryl, thio substituted aryl, thioalkoxy, or halo;

one of Z₅ and Z₆ is a protected hydroxyl group and the other of Z₅ and Z₆ is —O-(L)ₙ-sm;

L is a linking moiety;

n is 0 or 1; and

sm is a support medium;

treating said reactive hydroxyl group with a first monomeric subunit having an activated phosphorus group and a further protected hydroxyl group thereon for a time and under conditions sufficient to form a monomer-functionalized support medium.

In certain embodiments, the method may further comprise the steps of:

treating said monomer-functionalized support medium with a capping agent; and

optionally, treating said monomer-functionalized support medium with an oxidizing agent.

In other embodiments, the method includes the further steps of:

deblocking said further protected hydroxyl group to give a reactive hydroxyl group;

treating the reactive hydroxyl group with a further monomeric subunit having an activated phosphorus group and a further protected hydroxyl group thereon for a time and under conditions sufficient to form an extended compound;

treating said extended compound with a capping agent;

optionally, treating said extended compound with an oxidizing or sulfurizing agent;

repeating the preceding four steps one or more times to form a further extended compound; and

treating said further extended compound with an oxidizing or sulfurizing agent to form an oligomeric compound.

Preferably, said last treating step is effective to remove protecting groups present on said oligomeric compound. Preferably, said cleaved oligomeric compound has a terminal hydroxyl group at the site of cleavage and, more preferably, said terminal hydroxyl group is attached to a 2′- or 3′-position of a nucleoside that is located at the 3′-terminus of said oligomeric compound, preferably at the 3′-position.

In certain other embodiments, the process further comprises the step of treating said oligomeric compound with a reagent effective to cleave said oligomeric compound from said support medium. Preferably, said treating step is effective to remove protecting groups present on said oligomeric compound. Preferably, said cleaved oligomeric compound has a terminal hydroxyl group at the site of cleavage and, more preferably, said terminal hydroxyl group is attached to a 2′- or 3′-position of a nucleoside that is located at the 3′-terminus of said oligomeric compound.

Preferably, the treating step of said reactive hydroxyl group with a monomeric subunit having an activated phosphorus group and a further protected hydroxyl is performed in the presence of an activating agent.

Preferably, said monomeric subunit having an activated phosphorus group is a phosphoramidite, an H-phosphonate and a phosphate triester.

Preferably, said hydroxyl protecting group Z₃ and each of said further hydroxyl protecting groups are acid labile.

In certain preferred embodiments of the process, said hydroxyl protecting group Z₃ and each of said further hydroxyl protecting groups are removed by contacting said hydroxyl protecting groups with an acid, wherein said acid is formic acid, acetic acid, chloroacetic acid, dichloroacetic acid, trichloroacetic acid, trifluoroacetic acid, benzenesulfonic acid, toluenesulfonic acid, or phenylphosphoric acid.

Preferably, the oligomeric compounds are oligonucleotides, modified oligonucleotides, oligonucleotide analogs, oligonucleosides, oligonucleotide mimetics, hemimers, gapmers or chimeras.

The hydroxyl-protecting group can be removed from the compounds of the invention by techniques well known in the art to form the free hydroxyl. For example, dimethoxytrityl protecting groups can be removed by protic acids such as formic acid, dichloroacetic acid, trichloroacetic acid, p-toluene sulphonic acid or with Lewis acids such as for example zinc bromide. See, for example, Greene and Wuts, supra.

The oligomeric compounds prepared in accordance with the process of the invention may be conveniently and routinely made through the well-known technique of support-based synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Preferably, the oligomeric compounds prepared by the process of the invention utilize phosphoramidite chemistry on the support medium. The phosphoramidites can modified at the heterocyclic base, the sugar or both positions to enable the synthesis of fully modified positionally modified oligonucleotides and their analogs.

Conventional iterative solid phase oligonucleotide synthetic regimes are utilized to synthesize the oligomeric compounds of the invention. Representative support-based techniques are those typically employed for DNA and RNA synthesis utilizing standard phosphoramidite chemistry, (see, e.g., Protocols For Oligonucleotides And Analogs, Agrawal, S., ed., Humana Press, Totowa, N.J., 1993, hereby incorporated by reference in its entirety). Further details of methods useful for preparing oligonucleotides may be found in Sekine, M., et al., J. Org. Chem., 1979, 44, 2325; Dahl, O., Sulfur Reports, 1991, 11, 167-192; Kresse, J., et. al., Nucleic Acids Research, 1975, 2, 1-9; Eckstein, F., Ann. Rev. Biochem., 1985, 54, 367-402; and U.S. Pat. No. 5,210,264.

A preferred synthetic solid phase synthesis of oligonucleotides utilizes phosphoramidites as activated phosphate compounds. In this technique, a phosphoramidite monomer is reacted with a free hydroxyl on the growing oligomer chain to produce an intermediate phosphite compound, which is subsequently oxidized to the P^{V} state using standard methods. This technique is commonly used for the synthesis of several types of linkages including phosphodiester, phosphorothioate, and phosphorodithioate linkages.

The phosphite triester linkage is subsequently oxidized or sulfurized. Choice of oxidizing or sulfurizing agent will determine whether the linkage will be oxidized or sulfurized to a phosphotriester, thiophosphotriester, or a dithiophosphotriester linkage.

It is generally preferable to perform a capping step, either prior to or after oxidation or sulfurization of the phosphite triester, thiophosphite triester, or dithiophosphite triester. Such a capping step is generally known to be beneficial by preventing shortened oligomer chains, by blocking chains that have not reacted in the coupling cycle. One representative reagent used for capping is acetic anhydride. Other suitable capping reagents and methodologies can be found in U.S. Pat. No. 4,816,571, hereby incorporated by reference in its entirety. Treatment with an acid removes the 5′-hydroxyl protecting group, and the synthetic cycle is repeated until the desired oligomer is achieved.

A representative list of capping reagents useful in the process of the present invention include without limitation, acetic anhydride, t-butylphenoxyacetic anhydride, phosphite monoesters, and selected acid chlorides preferably delivered concurrently with a nucleophilic catalyst (e.g. a strong base) such as for example dimethylaminopyridine, N-methylimidazole or triethylamine. Generally capping reagents comprise a mixture of Cap A and Cap B. Representative mixtures include without limitation:

Cap A: acetic anhydride in acetonitrile or tetrahydrofuran; chloroacetic anhydride in acetonitrile or tetrahydrofuran;

Cap B: N-methylimidazole and pyridine in acetonitrile or tetrahydrofuran; 4-dimethylaminopyridine (DMAP) and pyridine in acetonitrile or tetrahydrofuran; 2,6-lutidine and N-methylimidazole in acetonitrile or tetrahydrofuran.

A more detailed description capping reagents is discussed in U.S. Pat. No. 4,816,571, issued Mar. 28, 1989, which is incorporated herein by reference. A preferred capping reagent is acetic anhydride routinely used as a mixture of cap A and cap B.

Useful sulfurizing agents include Beaucage reagent described in e.g., Iyer et al., J. Am. Chem. Soc., 112, 1253-1254 (1990); and Iyer et al., J Org Chem, 55, 4693-4699 (1990); tetraethyl-thiuram disulfide as described in Vu et al., Tetrahedron Lett., 32, 3005-3007 (1991); dibenzoyl tetrasulfide as described in Rao et al., Tetrahedron Lett., 33, 4839-4842 (1992); di(phenylacetyl)disulfide, as described in Kamer, et al., Tetrahedron Lett., 30, 6757-6760 (1989); bis(O,O-diisopropoxy phosphinothioyl)disulfide, Wojciech J. Stec., Tetrahedron Lett., 1993, 34, 5317-5320; sulfur; and sulfur in combination with ligands like triaryl, trialkyl or triaralkyl or trialkaryl phosphines. Useful oxidizing agents, in addition to those set out above, include iodine/tetrahydrofuran/water/pyridine; hydrogen peroxide/water; tert-butyl hydroperoxide; or a peracid like m-chloroperbenzoic acid. In the case of sulfurization, the reaction is performed under anhydrous conditions with the exclusion of air, in particular oxygen; whereas, in the case of oxidation the reaction can be performed under aqueous conditions.

The internucleoside linkages of the oligonucleotides described herein, can be any internucleoside linkage as is known in the art, including phosphorus based linking groups, such as phosphite, phosphodiester, phosphorothioate, and phosphorodithioate linkages. Such linkages can be protected, i.e., they can bear, for example, phosphorus-protecting groups. As used herein, the term “phosphorus protecting group” is intended to denote protecting groups that are known to be useful to protect phosphorus-containing linkages during oligonucleotide synthesis. One such preferred phosphorus-protecting group is the P-cyanoethyl protecting group.

Other representative phosphorus protecting groups include —CH₂CH═CHCH₂CN, para-C₆H₄CH₂CN, —(CH₂)₂-5-N(H)COCF₃, —CH₂CH₂Si(C₆H₅)₂CH₂, —CH₂CH₂N(CH₃)COCF₃ and others known in the art.

The processes of the present invention illustrate the use of activated phosphorus compounds (e.g., compounds having activated phosphorus-containing substituent groups) in coupling reactions. As used herein, the term “activated phosphorus compounds” includes monomers and oligomers that have an activated phosphorus-containing substituent group that is reactive with a hydroxyl group of another monomeric or oligomeric compound to form a phosphorus-containing internucleotide linkage. Such activated phosphorus groups contain activated phosphorus atoms in P^{III} valence state and are known in the art and include, but are not limited to, phosphoramidite, H-phosphonate, phosphate triesters and chiral auxiliaries. A preferred synthetic solid phase synthesis utilizes phosphoramidites as activated phosphates. The phosphoramidites utilize P^{III} chemistry. The intermediate phosphite compounds are subsequently oxidized to the P^{V} state using known methods to yield, in a preferred embodiment, phosphodiester or phosphorothioate internucleotide linkages. Additional activated phosphates and phosphites are disclosed in Tetrahedron Report Number 309 (Beaucage and Iyer, Tetrahedron, 1992, 48, 2223-2311).

Activated phosphorus groups are useful in the preparation of a wide range of oligomeric compounds including but not limited to oligonucleosides and oligonucleotides as well as oligonucleotides that have been modified or conjugated with other groups at the base or sugar or both. Also included are oligonucleotide mimetics including but not limited to peptide nucleic acids (PNA), morpholino nucleic acids, cyclohexenyl nucleic acids (CeNA), anhydrohexitol nucleic acids, locked nucleic acids (LNA), bicyclic and tricyclic nucleic acids, phosphonomonoester nucleic acids and cyclobutyl nucleic acids. A representative example of one type of oligomer synthesis that utilizes the coupling of an activated phosphorus group with a reactive hydroxyl group is the widely used phosphoramidite approach. A phosphoramidite monomeric subunit is reacted under appropriate conditions with a reactive hydroxyl group to form a phosphite linkage that is further oxidized to a phosphodiester or phosphorothioate linkage. This approach commonly utilizes nucleoside phosphoramidites of the formula:

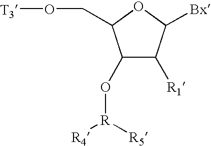

wherein

each Bx′ is an optionally protected heterocyclic base moiety;

each R_{1′} is, independently, H or an optionally protected sugar substituent group;

T_{3′} is an hydroxyl protecting group, a nucleoside, a nucleotide, an oligonucleoside or an oligonucleotide;

R_{4′} is N(L₁)L₂;

each L₁ and L₂ is, independently, C₁₋₆ alkyl;

or L₁ and L₂ are joined together to form a 4- to 7-membered heterocyclic ring system including the nitrogen atom to which L1 and L2 are attached, wherein said ring system optionally includes at least one additional heteroatom, wherein said heteroatom is O, N or S;

R_{5′} is X₁;

X₁ is Pg-O—, Pg-S—, C₁₋₁₀ straight or branched chain alkyl, CH₃(CH₂)₀₋₁₀—O— or —NR_{6′}R_{7′};

Pg is a protecting/blocking group; and

each R_{6′} and R_{7′} is, independently, hydrogen, C₁₋₁₀ alkyl, cycloalkyl or aryl;

or optionally, R_{6′} and R_{7′}, together with the nitrogen atom to which they are attached form a cyclic moiety that may include an additional heteroatom, wherein said heteroatom is O, S and N; or

R_{4′} and R_{5′} together with the phosphorus atom to which R_{4′} and R_{5′} are attached form a chiral auxiliary.

Groups that are attached to the phosphorus atom of internucleotide linkages before and after oxidation (R_{4′} and R_{5′}) can include nitrogen containing cyclic moieties such as morpholine. Such oxidized internucleoside linkages include a phosphoromorpholidothioate linkage (Wilk et al., Nucleosides and Nucleotides, 1991, 10, 319-322). Further cyclic moieties amenable to the present invention include mono-, bi- or tricyclic ring moieties which may be substituted with groups such as oxo, acyl, alkoxy, alkoxycarbonyl, alkyl, alkenyl, alkynyl, amino, amido, azido, aryl, heteroaryl, carboxylic acid, cyano, guanidino, halo, haloalkyl, haloalkoxy, hydrazino, ODMT, alkylsulfonyl, nitro, sulfide, sulfone, sulfonamide, thiol and thioalkoxy. A preferred bicyclic ring structure that includes nitrogen is phthalimido.

Some representative examples of R_{4′} and R_{5′} groups that are known to the art skilled and are amenable to the present invention are shown below:

| R_{4′} | R_{5′} |
|---|---|
| 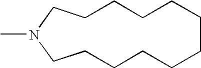 | —O—CH₃ |
| 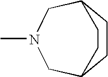 | —O—CH₃ |
| 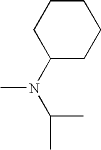 | —O—CH₃ |
| 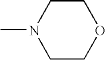 | 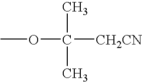 |
| 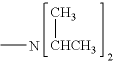 | —O—CH₂CH₂SiCH₃ |
| 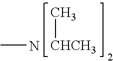 | 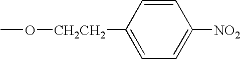 |
| —N(CH₃)₂ | 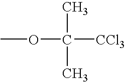 |
| —N(CH₂CH₃)₂ | 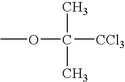 |
| 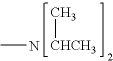 | 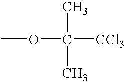 |
| 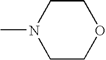 | 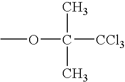 |
| —N(CH₃)₂ | —O—CH₂CCl₃ |
| 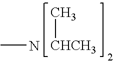 | —CH₂CH═CH₂ |
| 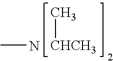 | —O—CH₂CH₂CN |

Further examples include:

| R4′ | R5′ |
|---|---|
| —N(CH₃)₂ | 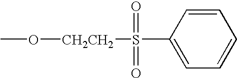 |
| 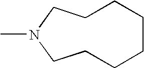 | 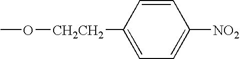 |
| 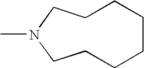 | 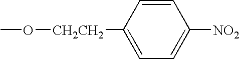 |
| 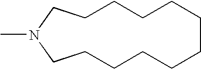 | 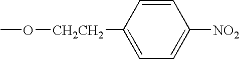 |
| 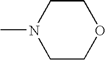 | 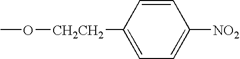 |
| 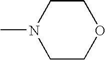 | 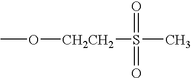 |
| 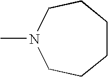 | 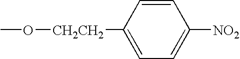 |
| 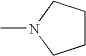 | —O—CH₃ |
| 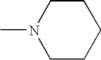 | —O—CH₃ |
| 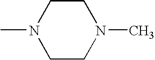 | —O—CH₃ |
| 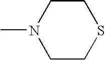 | —O—CH₃ |
| 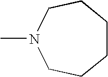 | —O—CH₃ |
| 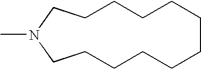 | —O—CH₃ |

Representative nucleobases useful in the methods and conjugated oligomeric compounds of the invention include adenine, guanine, cytosine, uridine, and thymine, as well as other non-naturally occurring and natural nucleobases such as xanthine, hypoxanthine, 2-aminoadenine, 6-methyl and other alkyl derivatives of adenine and guanine, 2-propyl and other alkyl derivatives of adenine and guanine, 5-halo uracil and cytosine, 6-azo uracil, cytosine and thymine, 5-uracil (pseudo uracil), 4-thiouracil, 8-halo, oxa, amino, thiol, thioalkyl, hydroxyl and other 8-substituted adenines and guanines, 5-trifluoromethyl and other 5-substituted uracils and cytosines, 7-methylguanine. Further naturally and non naturally occurring nucleobases include those disclosed in U.S. Pat. No. 3,687,808, in Chapter 15 by Sanghvi, in Antisense Research and Application, Ed. S. T. Crooke and B. Lebleu, CRC Press, 1993, in Englisch et al., Angewandte Chemie, International Edition, 1991, 30, 613-722 (see especially pages 622 and 623, and in the Concise Encyclopedia of Polymer Science and Engineering, J. I. Kroschwitz Ed., John Wiley & Sons, 1990, pages 858-859, Cook, P. D., Anti-Cancer Drug Design, 1991, 6, 585-607, each of which are hereby incorporated by reference in their entirety. The term “nucleosidic base” is further intended to include heterocyclic compounds that can serve as like nucleosidic bases including certain “universal bases” that are not nucleosidic bases in the most classical sense but serve as nucleosidic bases. Especially mentioned as a universal base is 3-nitropyrrole.

Representative 2′-sugar modifications amenable to the present invention include fluoro, O-alkyl, O-alkylamino, O-alkylalkoxy, protected O-alkylamino, O-alkylaminoalkyl, O-alkyl imidazole, and polyethers of the formula (O-alkyl)ₘ, where m is 1 to about 10. Preferred among these polyethers are linear and cyclic polyethylene glycols (PEGs), and (PEG)-containing groups, such as crown ethers and those which are disclosed by Ouchi, et al., Drug Design and Discovery, 1992, 9, 93, Ravasio, et al., J. Org. Chem. 1991, 56, 4329, and Delgardo et. al., Critical Reviews in Therapeutic Drug Carrier Systems 1992, 9, 249, each of which are hereby incorporated by reference in their entirety. Further sugar modifications are disclosed in Cook, P. D., supra. Fluoro, O-alkyl, O-alkylamino, O-alkyl imidazole, O-alkylaminoalkyl, and alkyl amino substitution is described in U.S. Pat. No. 6,166,197, hereby incorporated by reference in its entirety.

Sugars having O-substitutions on the ribosyl ring are also amenable to the present invention. Representative substitutions for ring 0 include S, CH₂, CHF, and CF₂, see, e.g., Secrist, et al., Abstract 21, Program & Abstracts, Tenth International Roundtable, Nucleosides, Nucleotides and their Biological Applications, Park City, Utah, Sep. 16-20, 1992, hereby incorporated by reference in its entirety.

Representative hydroxyl protecting groups commonly used in the art may be found in Beaucage, et al., Tetrahedron 1992, 48, 2223; and Greene and Wuts, Protective Groups in Organic Synthesis, Chapter 2, 2d ed, John Wiley & Sons, New York, 1991, each of which are hereby incorporated by reference in their entirety. Preferred protecting groups include dimethoxytrityl (DMT), monomethoxytrityl, 9-phenylxanthen-9-yl (Pixyl) and 9-(p-methoxyphenyl)xanthen-9-yl (Mox). The protecting group can be removed from oligonucleotides of the conjugated oligomeric compound of the invention by techniques well known in the art to form the free hydroxyl. For example, dimethoxytrityl protecting groups can be removed by protic acids such as formic acid, dichloroacetic acid, trichloroacetic acid, p-toluene sulphonic acid or with Lewis acids such as for example zinc bromide. See, for example, Greene and Wuts, supra.

In some preferred embodiments of the invention amino groups are appended to alkyl or to other groups such as, for example, to 2′-alkoxy groups. Such amino groups are also commonly present in naturally occurring and non-naturally occurring nucleobases. It is generally preferred that these amino groups be in protected form during the synthesis of oligonucleotides of the invention. Representative amino protecting groups suitable for these. purposes are discussed in Greene and Wuts, Protective Groups in Organic Synthesis, Chapter 7, 2d ed, John Wiley & Sons, New York, 1991. Generally, as used herein, the term “protected” when used in connection with a molecular moiety such as “nucleobase” indicates that the molecular moiety contains one or more functionalities protected by protecting groups.

In the context of this specification, alkyl (generally C₁-C₂₀), alkenyl (generally C₂-C₂₀), and alkynyl (generally C₂-C₂₀) groups include but are not limited to substituted and unsubstituted straight chain, branch chain, and alicyclic hydrocarbons, including methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl and other higher carbon alkyl groups. Further examples include 2-methylpropyl, 2-methyl-4-ethylbutyl, 2,4-diethylbutyl, 3-propylbutyl, 2,8-dibutyldecyl, 6,6-dimethyloctyl, 6-propyl-6-butyloctyl, 2-methylbutyl, 2-methylpentyl, 3-methylpentyl, 2-ethylhexyl and other branched chain groups, allyl, crotyl, propargyl, 2-pentenyl and other unsaturated groups containing a pi bond, cyclohexane, cyclopentane, adamantane as well as other alicyclic groups, 3-penten-2-one, 3-methyl-2-butanol, 2-cyanooctyl, 3-methoxy-4-heptanal, 3-nitrobutyl, 4-isopropoxydodecyl, 4-azido-2-nitrodecyl, 5-mercaptononyl, 4-amino-1-pentenyl as well as other substituted groups. Representative alkyl substituents are disclosed in U.S. Pat. No. 5,212,295, at column 12, lines 41-50, hereby incorporated by reference in its entirety.

Further, in the context of this invention, a straight chain compound means an open chain compound, such as an aliphatic compound, including alkyl, alkenyl, or alkynyl compounds; lower alkyl, alkenyl, or alkynyl as used herein include but are not limited to hydrocarbyl compounds from about 1 to about 6 carbon atoms. A branched compound, as used herein, comprises a straight chain compound, such as an alkyl, alkenyl, alkynyl compound, which has further straight or branched chains attached to the carbon atoms of the straight chain. A cyclic compound, as used herein, refers to closed chain compounds, i.e. a ring of carbon atoms, such as an alicyclic or aromatic compound. The straight, branched, or cyclic compounds may be internally interrupted, as in alkoxy or heterocyclic compounds. In the context of this invention, internally interrupted means that the carbon chains may be interrupted with heteroatoms such as O, N, or S. However, if desired, the carbon chain may have no heteroatoms.

Preferably, the process of the invention further comprises the step of treating said oligomeric compound with a reagent effective to cleave said oligomeric compound from said support medium. Preferred cleaving reagents include gaseous ammonia, alkylamines including methylamine, ethylamine, or propylamine, solutions of ammonia, alkylamines including methylamine, ethylamine, propylamine, t-butylamine, piperidine, pyrrolidine, piperazine in water or organic solvents, solutions of alkalis, lithium hydroxide, sodium hydroxide, potassium hydroxide in water or organic solvents including methanol, ethanol, propanol, or isopropanol, solutions of lithium carbonate, sodium carbonate, or potassium carbonate in water or organic solvents including methyl alcohol, or ethyl alcohol.

Preferably, the process further comprises the step of treating said oligomeric compound with a reagent effective to remove protecting groups from said oligomeric compound. Preferred deprotecting reagents include gaseous ammonia, alkylamines including methylamine, ethylamine, or propylamine, solutions of ammonia, alkylamines including methylamine, ethylamine, propylamine, t-butylamine, piperidine, pyrrolidine, piperazine in water or organic solvents, solutions of alkalies lithium hydroxide, sodium hydroxide, potassium hydroxide in water or organic solvents including methanol, ethanol, propanol, or isopropanol, solutions of lithium carbonate, sodium carbonate, or potassium carbonate in water or organic solvents including methyl alcohol, or ethyl alcohol.

The present invention is also useful for the preparation of oligomeric compounds incorporating at least one 2′-O-protected nucleoside. After incorporation and appropriate deprotection the 2′-O-protected nucleoside will be converted to a ribonucleoside at the position of incorporation. The number and position of the 2-ribonucleoside units in the final oligomeric compound can vary from one at any site or the strategy can be used to prepare up to a full 2′-OH modified oligomeric compound. All 2′-O-protecting groups amenable to the synthesis of oligomeric compounds are included in the present invention. In general a protected nucleoside is attached to a solid support by for example a succinate linker. Then the oligonucleotide is elongated by repeated cycles of deprotecting the 5′-terminal hydroxyl group, coupling of a further nucleoside unit, capping and oxidation (alternatively sulfurization). In a more frequently used method of synthesis the completed oligonucleotide is cleaved from the solid support with the removal of phosphate protecting groups and exocyclic amino protecting groups by treatment with an ammonia solution. Then a further deprotection step is normally required for the more specialized protecting groups used for the protection of 2′-hydroxyl groups which will give the fully deprotected oligonucleotide.

A large number of 2′-O-protecting groups have been used for the synthesis of oligoribonucleotides but over the years more effective groups have been discovered. The key to an effective 2′-O-protecting group is that it is capable of selectively being introduced at the 2′-O-position and that it can be removed easily after synthesis without the formation of unwanted side products. The protecting group also needs to be inert to the normal deprotecting, coupling, and capping steps required for oligoribonucleotide synthesis. Some of the protecting groups used initially for oligoribonucleotide synthesis included tetrahydropyran-1yl and 4-methoxytetrahydropyran-4-yl. These two groups are not compatible with all 5′-O-protecting groups so modified versions were used with 5′-DMT groups such as 1-(2-fluorophenyl)-4-methoxypiperidin-4-yl (Fpmp). Reese has identified a number of piperidine derivatives (like Fpmp) that are useful in the synthesis of oligoribonucleotides including 1-[(chloro-4-methyl)phenyl]-4′-methoxypiperidin-4-yl (Reese et al., Tetrahedron Lett., 1986, (27), 2291). Another approach was to replace the standard 5′-DMT (dimethoxytrityl) group with protecting groups that were removed under non-acidic conditions such as levulinyl and 9-fluorenylmethoxycarbonyl. Such groups enable the use of acid labile 2′-protecting groups for oligoribonucleotide synthesis. Another more widely used protecting group initially used for the synthesis of oligoribonucleotides was the t-butyldimethylsilyl group (Ogilvie et al., Tetrahedron Lett., 1974, 2861; Hakimelahi et al., Tetrahedron Lett., 1981, (22), 2543; and Jones et al., J. Chem. Soc. Perkin I., 2762). The 2′-O-protecting groups can require special reagents for their removal such as for example the t-butyldimethylsilyl group is normally removed after all other cleaving/deprotecting steps by treatment of the oligomeric compound with tetrabutylammonium fluoride (TBAF).

One group of researchers examined a number of 2′-O-protecting groups (Pitsch, S., Chimia, 2001, (55), 320-324.) The group examined fluoride labile and photolabile protecting groups that are removed using moderate conditions. One photolabile group that was examined was the [2-(nitrobenzyl)oxy]methyl (nbm) protecting group (Schwartz et al., Bioorg. Med. Chem. Lett., 1992, (2), 1019.) Other groups examined included a number structurally related formaldehyde acetal-derived, 2′-O-protecting groups. Also prepared were a number of related protecting groups for preparing 2′-O-alkylated nucleoside phosphoramidites including 2′-O—[(triisopropylsilyl)oxy]methyl (2′-O—CH₂—O—Si(iPr)₃, TOM). One 2′-O-protecting group that was prepared to be used orthogonally to the TOM group was 2′-O—[(R)-1-(2-nitrophenyl)ethyloxy)methyl]((R)-mnbm).

Another strategy using a fluoride labile 5′-O-protecting group (non-acid labile) and an acid labile 2′-O-protecting group has been reported (Scaringe, Stephen A., Methods, 2001, (23) 206-217). A number of possible silyl ethers were examined for 5′-O-protection and a number of acetals and orthoesters were examined for 2′-O-protection. The protection scheme that gave the best results was 5′-O-silyl ether-2′-ACE (5′-O-bis(trimethylsiloxy)cyclododecyloxysilyl ether (DOD)-2′-O-bis(2-acetoxyethoxy)methyl (ACE). This approach uses a modified phosphoramidite synthesis approach in that some different reagents are required that are not routinely used for RNA/DNA synthesis.

Although a lot of research has focused on the synthesis of oligoribonucleotides the main RNA synthesis strategies that are presently being used commercially include 5′-O-DMT-2′-O-t-butyldimethylsilyl (TBDMS), 5′-O-DMT-2′-O-[1(2-fluorophenyl)-4-methoxypiperidin-4-yl] (FPMP), 2′-O-[(triisopropylsilyl)oxy]methyl (2′-O—CH₂—O—Si(iPr)₃ (TOM), and the 5′-O-silyl ether-2′-ACE (5′-O-bis(trimethylsiloxy)cyclododecyloxysilyl ether (DOD)-2′-O-bis(2-acetoxyethoxy)methyl (ACE). A current list of some of the major companies currently offering RNA products include Pierce Nucleic Acid Technologies, Dharmacon Research Inc., Ameri Biotechnologies Inc., and Integrated DNA Technologies, Inc. One company, Princeton Separations, is marketing an RNA synthesis activator advertised to reduce coupling times especially with TOM and TBDMS chemistries. Such an activator would also be amenable to the present invention.

The structures corresponding to these protecting groups are shown below.

5′-O-DMT-2′-O-t-butyldimethylsilyl (TBDMS):

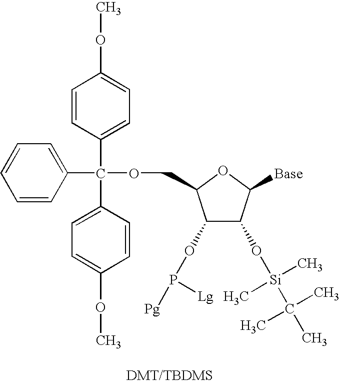

5′-O-DMT-2′-O-[1(2-fluorophenyl)-4-methoxypiperidin-4-yl] (FPMP):

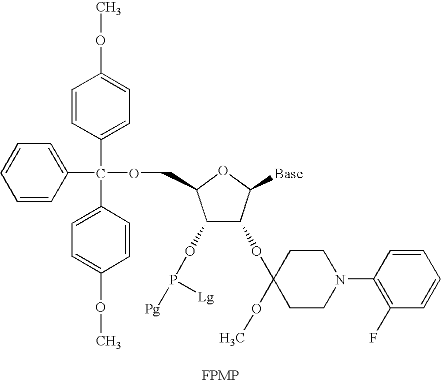

2′-O-[(triisopropylsilyl)oxy]methyl (2′-O—CH₂—O—Si(iPr)₃ (TOM):

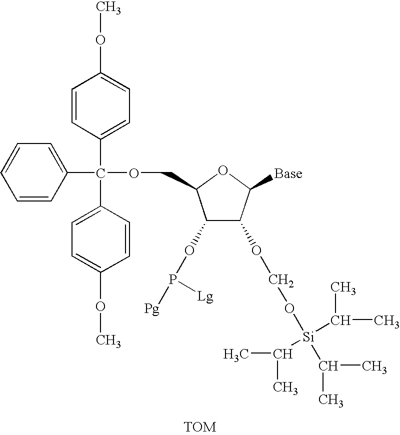

and 5′-O-bis(trimethylsiloxy)cyclododecyloxysilyl ether-2′-O-bis(2-acetoxyethoxy)methyl:

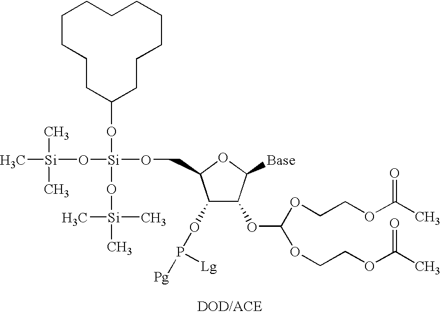

All of the aforementioned RNA synthesis strategies are amenable to the present invention. Strategies that would be a hybrid of the above e.g. using a 5′-protecting group from one strategy with a 2′-O-protecting from another strategy is also amenable to the present invention.

The preparation of ribonucleotides and oligomeric compounds having at least one ribonucleoside incorporated and all the possible configurations falling in between these two extremes are encompassed by the present invention. The corresponding oligomeric comounds can be hybridized to further oligomeric compounds including oligoribonucleotides having regions of complementarity to form double-stranded (duplexed) oligomeric compounds. Such double stranded oligonucleotide moieties have been shown in the art to modulate target expression and regulate translation as well as RNA processsing via an antisense mechanism. Moreover, the double-stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double-stranded moieties have been shown to inhibit the target by the classical hybridization of antisense strand of the duplex to the target, thereby triggering enzymatic degradation of the target (Tijsterman et al., Science, 2002, 295, 694-697).

The methods of preparing oligomeric compounds of the present invention can also be applied in the areas of drug discovery and target validation. The present invention comprehends the use of the oligomeric compounds and preferred targets identified herein in drug discovery efforts to elucidate relationships that exist between proteins and a disease state, phenotype, or condition. These methods include detecting or modulating a target peptide comprising contacting a sample, tissue, cell, or organism with the oligomeric compounds of the present invention, measuring the nucleic acid or protein level of the target and/or a related phenotypic or chemical endpoint at some time after treatment, and optionally comparing the measured value to a non-treated sample or sample treated with a further oligomeric compound of the invention. These methods can also be performed in parallel or in combination with other experiments to determine the function of unknown genes for the process of target validation or to determine the validity of a particular gene product as a target for treatment or prevention of a particular disease, condition, or phenotype.

Effect of nucleoside modifications on RNAi activity is evaluated according to existing literature (Elbashir et al., Nature (2001), 411, 494-498; Nishikura et al., Cell (2001), 107, 415-416; and Bass et al., Cell (2000), 101, 235-238.)

Following assembly of the desired oligomeric compound, the next step will normally be deprotection of the oligomeric compound and cleavage of the synthesized oligomeric compound from the support medium. These processes can take place substantially simultaneously, thereby providing the free oligomeric compound in the desired form.

The support media useful with the compounds and in the processes of the invention are used for attachment of a first nucleoside or other monomeric subunit that is then iteratively elongated to give a final oligomeric compound. Support media may be selected to be insoluble or have variable solubility in different solvents to allow the growing support bound polymer to be either in or out of solution as desired. Traditional support media such as solid supports are generally insoluble and are routinely placed in a reaction vessel while reagents and solvents react and or wash the growing chain until cleavage the final polymeric compound. More recent approaches have introduced soluble supports including soluble polymer supports to allow precipitating and dissolving the iteratively synthesized product at desired points in the synthesis (Gravert et al., Chem. Rev., 1997, 97, 489-510).

The current method of choice for the preparation of oligomeric compounds utilizes support media. Support media is used for attachment of a first nucleoside or other monomeric subunit that is then iteratively elongated to give a final oligomeric compound or other polymer such as a polypeptide. Support media can be selected to be insoluble or have variable solubility in different solvents to allow the growing support bound polymer to be either in or out of solution as desired. Traditional support media such as solid supports are for the most part insoluble and are routinely placed in a reaction vessel while reagents and solvents react and or wash the growing chain until cleavage the final polymeric compound. More recent approaches have introduced soluble supports including soluble polymer supports to allow precipitating and dissolving the iteratively synthesized product at desired points in the synthesis (Gravert et al., Chem. Rev., 1997, 97, 489-510).

The term “support media” is intended to include all forms of support known to the art skilled for the synthesis of oligomeric compounds and related compounds such as peptides. Some representative support media that are amenable to the methods of the present invention include but are not limited to the following: controlled pore glass (CPG); oxalyl-controlled pore glass (see, e.g., Alul, et al., Nucleic Acids Research 1991, 19, 1527); silica-containing particles, such as porous glass beads and silica gel such as that formed by the reaction of trichloro-[3-(4-chloromethyl)phenyl]propylsilane and porous glass beads (see Parr and Grohmann, Angew. Chem. Internal. Ed. 1972, 11, 314, sold under the trademark “PORASIL E” by Waters Associates, Framingham, Mass., USA); the mono ester of 1,4-dihydroxymethylbenzene and silica (see Bayer and Jung, Tetrahedron Lett., 1970, 4503, sold under the trademark “BIOPAK” by Waters Associates); TENTAGEL (see, e.g., Wright, et al., Tetrahedron Letters 1993, 34, 3373); cross-linked styrene/divinylbenzene copolymer beaded matrix or POROS, a copolymer of polystyrene/divinylbenzene (available from Perceptive Biosystems); soluble support media, polyethylene glycol PEG's (see Bonora et al., Organic Process Research & Development, 2000, 4, 225-231).

Further support media amenable to the present invention include without limitation PEPS support a polyethylene (PE) film with pendant long-chain polystyrene (PS) grafts (molecular weight on the order of 10⁶, (see Berg, et al., J. Am. Chem. Soc., 1989, 111, 8024 and International Patent Application WO 90/02749),). The loading capacity of the film is as high as that of a beaded matrix with the additional flexibility to accomodate multiple syntheses simultaneously. The PEPS film may be fashioned in the form of discrete, labeled sheets, each serving as an individual compartment. During all the identical steps of the synthetic cycles, the sheets are kept together in a single reaction vessel to permit concurrent preparation of a multitude of peptides at a rate close to that of a single peptide by conventional methods. Also, experiments with other geometries of the PEPS polymer such as, for example, non-woven felt, knitted net, sticks or microwellplates have not indicated any limitations of the synthetic efficacy.

Further support media amenable to the present invention include without limitation particles based upon copolymers of dimethylacrylamide cross-linked with N,N′-bisacryloylethylenediamine, including a known amount of N-tertbutoxycarbonyl-beta-alanyl-N′-acryloylhexamethylenediamine. Several spacer molecules are typically added via the beta alanyl group, followed thereafter by the amino acid residue subunits. Also, the beta alanyl-containing monomer can be replaced with an acryloyl safcosine monomer during polymerization to form resin beads. The polymerization is followed by reaction of the beads with ethylenediamine to form resin particles that contain primary amines as the covalently linked functionality. The polyacrylamide-based supports are relatively more hydrophilic than are the polystyrene-based supports and are usually used with polar aprotic solvents including dimethylformamide, dimethylacetamide, N-methylpyrrolidone and the like (see Atherton, et al., J. Am. Chem. Soc., 1975, 97, 6584, Bioorg. Chem. 1979, 8, 351, and J. C. S. Perkin I 538 (1981)).

Further support media amenable to the present invention include without limitation a composite of a resin and another material that is also substantially inert to the organic synthesis reaction conditions employed. One exemplary composite (see Scott, et al., J. Chrom. Sci., 1971, 9, 577) utilizes glass particles coated with a hydrophobic, cross-linked styrene polymer containing reactive chloromethyl groups, and is supplied by Northgate Laboratories, Inc., of Hamden, Conn., USA. Another exemplary composite contains a core of fluorinated ethylene polymer onto which has been grafted polystyrene (see Kent and Merrifield, Israel J. Chem. 1978, 17, 243 and van Rietschoten in Peptides 1974, Y. Wolman, Ed., Wiley and Sons, New York, 1975, pp. 113-116). Contiguous solid supports other than PEPS, such as cotton sheets (Lebl and Eichler, Peptide Res. 1989, 2, 232) and hydroxypropylacrylate-coated polypropylene membranes (Daniels, et al., Tetrahedron Lett. 1989, 4345). Acrylic acid-grafted polyethylene-rods and 96-microtiter wells to immobilize the growing peptide chains and to perform the compartmentalized synthesis may be utilized. (Geysen, et al., Proc. Natl. Acad. Sci. USA, 1984, 81, 3998). A “tea bag” containing traditionally-used polymer beads. (Houghten, Proc. Natl. Acad. Sci. USA, 1985, 82, 5131). Simultaneous use of two different supports with different densities (Tregear, Chemistry and Biology of Peptides, J. Meienhofer, ed., Ann Arbor Sci. Publ., Ann Arbor, 1972 pp. 175-178). Combining of reaction vessels via a manifold (Gorman, Anal. Biochem., 1984, 136, 397). Multicolumn solid-phase synthesis (e.g., Krchnak, et al., Int. J. Peptide Protein Res., 1989, 33, 209), and Holm and Meldal, in “Proceedings of the 20th European Peptide Symposium”, G. Jung and E. Bayer, eds., Walter de Gruyter & Co., Berlin, 1989 pp. 208-210). Cellulose paper (Eichler, et al., Collect. Czech. Chem. Commun., 1989, 54, 1746). Support mediated synthesis of peptides have also been reported (see, Synthetic Peptides: A User's Guide, Gregory A. Grant, Ed. Oxford University Press 1992; U.S. Pat. Nos. 4,415,732; 4,458,066; 4,500,707; 4,668,777; 4,973,679; 5,132,418; 4,725,677 and Re-34,069.)

Support mediated synthesis of oligomeric compounds can also utilize a bifunctional linking moiety to attach the first compound or monomer to the support medium. In the simplest case a reactive functionality on the commercially available support medium forms a linkage with the first compound or monomeric sub unit. In a more complicated example a linker is used to react with the support medium and the first compound or monomeric sub unit. Regardless of how this first attachment is performed the support medium with the first compound or a universal linking compound, as per the present invention, is ready for the iterative coupling cycles of oligomer synthesis at this point. As used herein, “linking moiety” refers to a bifunctional linking group. In one aspect of the invention a linking moiety is used to attach a compound of formulas I, II or III to a support medium. A preferred linking group is —C(═O)—N(H)— where the carbonyl end is attached at position R1 or R2 of a compound of formula I, II or III and the amino end is attached to a support medium. Other bifunctional linking moieties known in the art will also function to attach compounds of the invention to a support medium. Such bifunctional linking moieties include but are not limited to succinyl and oxalyl groups. Other linking moieties include, but are not limited to, substituted or unsubstituted C₁-C₁₀ alkyl, substituted or unsubstituted C₂-C₁₀ alkenyl or substituted or unsubstituted C₂-C₁₀ alkynyl, wherein the substituent groups are selected from hydroxyl, amino, acyl, acetyl, alkoxy, carboxy, benzyl, phenyl, nitro, thiol, thioalkoxy, halogen, alkyl, aryl, alkenyl and alkynyl.

Support bound oligonucleotide synthesis relies on sequential addition of nucleotides to one end of a growing chain. Typically, a first nucleoside (having protecting groups on any exocyclic amine functionalities present) is attached to an appropriate glass bead support and activated phosphite compounds (typically nucleotide phosphoramidites, also bearing appropriate protecting groups) are added stepwise to elongate the growing oligonucleotide. Additional methods for solid-phase synthesis may be found in Caruthers U.S. Pat. Nos. 4,415,732; 4,458,066; 4,500,707; 4,668,777; 4,973,679; and 5,132,418; and Koster U.S. Pat. Nos. 4,725,677 and Re. 34,069.

Commercially available equipment routinely used for the support media based synthesis of oligomeric compounds and related compounds is sold by several vendors including, for example, Applied Biosystems (Foster City, Calif.). Any other means for such synthesis known in the art may additionally or alternatively be employed. Suitable solid phase techniques, including automated synthesis techniques, are described in F. Eckstein (ed.), Oligonucleotides and Analogues, A Practical Approach, Oxford University Press, New York (1991).

In some especially preferred embodiments, the nucleoside components of the oligomeric compounds are connected to each other by optionally protected phosphorothioate internucleoside linkages. Representative protecting groups for phosphorus containing internucleoside linkages such as phosphite, phosphodiester and phosphorothioate linages include β-cyanoethyl, diphenylsilylethyl, δ-cyanobutenyl, cyano p-xylyl (CPX), N-methyl-N-trifluoroacetyl ethyl (META), acetoxy phenoxy ethyl (APE) and butene-4-yl groups. See for example U.S. Pat. Nos. 4,725,677 and Re. 34,069 (β-cyanoethyl); Beaucage, S. L. and Iyer, R. P., Tetrahedron, 49 No. 10, pp. 1925-1963 (1993); Beaucage, S. L. and Iyer, R. P., Tetrahedron, 49 No. 46, pp. 10441-10488 (1993); Beaucage, S. L. and Iyer, R. P., Tetrahedron, 48 No. 12, pp. 2223-2311 (1992). Other representative phosphorus protecting groups include —CH₂CH═CHCH₂CN, para-C₆H₄CH₂CN, —(CH₂)₂-5-N(H)COCF₃, —CH₂CH₂Si(C₆H₅)₂CH₂, —CH₂CH₂N(CH₃)COCF₃ and others known in the art.

As used herein, the use in lists in methods or compositions of numbers and letters does not imply any specific sequence or priority, unless explicitly stated.

In a preferred embodiment, the oligomeric compounds produced using the reagents and by the processes of the invention may be administered in an effective amount to an organism to inhibit expression of a gene in the organism. Those skilled in the art would readily be able to determine the effective amount of the oligomeric compound based on the characteristics of the gene.

In another preferred embodiment, the oligomeric compound of the invention may be contacted in an effective amount to kill a pathogenic organism. Those skilled in the art would readily be able to determine the effective amount of the oligomeric compound to kill the organism.

The oligomeric compounds of the invention may be used in the therapeutic and/or prophylactic treatment of unicellular prokaryotic and multicellular eukaryotic organisms that utilize DNA-RNA transcription or RNA-protein transcription as a fundamental part of its hereditary, metabolic or cellular control. Such treatment may include the use of the oligomeric compounds of the invention in a method for killing a pathogenic organism, including viruses, bacteria and eukaryotic parasites.

The compositions of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The compositions of the invention encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the compositions of the invention, pharmaceutically acceptable salts of such prodrugs, and other bioequivalents. The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the oligomeric compounds of the invention: i.e., salts that retain the desired biological activity of the parent compound and do not impart undesired toxicological effects thereto. For oligonucleotides, preferred examples of pharmaceutically acceptable salts and their uses are further described in U.S. Pat. No. 6,287,860, which is incorporated herein in its entirety.

The present invention also includes pharmaceutical compositions and formulations which include the compositions of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration. Pharmaceutical compositions and formulations for topical administration may include transdernal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients.

Emulsions are typically heterogenous systems of one liquid dispersed in another in the form of droplets usually exceeding 0.1 μm in diameter. Emulsions may contain additional components in addition to the dispersed phases, and the active drug which may be present as a solution in either the aqueous phase, oily phase or itself as a separate phase. Microemulsions are included as an embodiment of the present invention. Emulsions and their uses are well known in the art and are further described in U.S. Pat. No. 6,287,860, which is incorporated herein in its entirety.

Formulations of the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

The pharmaceutical formulations and compositions of the present invention may also include surfactants. The use of surfactants in drug products, formulations and in emulsions is well known in the art. Surfactants and their uses are further described in U.S. Pat. No. 6,287,860, which is incorporated herein in its entirety.

In one embodiment, the present invention employs various penetration enhancers to effect the efficient delivery of nucleic acids, particularly oligonucleotides. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration.

Preferred formulations for topical administration include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, oligonucleotides of the invention may be encapsulated within liposomes or may form complexes thereto, in particular to cationic liposomes. Alternatively, oligonucleotides may be complexed to lipids, in particular to cationic lipids. Preferred fatty acids and esters, pharmaceutically acceptable salts thereof, and their uses are further described in U.S. Pat. No. 6,287,860, which is incorporated herein in its entirety. Topical formulations are described in detail in U.S. patent application Ser. No. 09/315,298 filed on May 20, 1999, which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts and fatty acids and their uses are further described in U.S. Pat. No. 6,287,860, which is incorporated herein in its entirety. Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally, in granular form including sprayed dried particles, or complexed to form micro or nanoparticles. Oligonucleotide complexing agents and their uses are further described in U.S. Pat. No. 6,287,860, which is incorporated herein in its entirety. Oral formulations for oligonucleotides and their preparation are described in detail in U.S. application Ser. No. 09/108,673 (filed Jul. 1, 1998), Ser. No. 09/315,298 (filed May 20, 1999) and Ser. No. 10/071,822, filed Feb. 8, 2002, each of which is incorporated herein by reference in their entirety.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Certain embodiments of the invention provide pharmaceutical compositions containing one or more of the compositions of the invention and one or more other chemotherapeutic agents which function by a non-antisense mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the compositions of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Combinations of compositions of the invention and other non-antisense drugs are also within the scope of this invention. One or more compositions of the invention can be used in combination with other therapeutic agents to create a coctail as is currently the strategy for certain viral infections.

In another related embodiment, therapeutically effective combination therapies may comprise the use of two or more compositions of the invention wherein the multiple compositions are targeted to a single or multiple nucleic acid targets. Numerous examples of antisense oligomeric compounds are known in the art. Two or more combined compounds may be used together or sequentially

The formulation of therapeutic compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 ug to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

While the present invention has been described with specificity in accordance with certain of its preferred embodiments, the following examples serve only to illustrate the invention and are not intended to limit the same.

The following examples are illustrative but are not meant to be limiting of the present invention.

### EXAMPLE 1

### (1α,2α,3α,4α,5α,6α)-5,6-dihydroxy-7-Oxabicyclo[2.2.1]heptane-2,3-dicarboxylic acid (2)

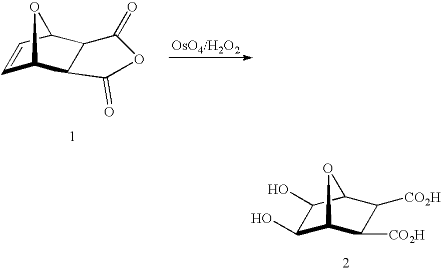

A solution of commercial (3aR,4S,7R,7aS)-rel-3a,4,7,7a-tetrahydro-4,7-epoxyiso-benzofuran-1,3-dione, 1, (9.05 g, 54.5 mmol) in hydrogen peroxide (30% aqueous, 2.97 g, 87.2 mmol), acetone (72.5 mL), ether (18.1 mL), and t-butanol (6.2 mL) was treated with osmium tetroxide (56 mg, 0.22 mmol) in t-butanol (2.86 mL) for 4 days at 28-30° C. The reaction mixture was treated with ether (90 mL) and kept at 4° C. for 1 h. The precipitate was filtered off, washed with ether and dried to give pure 2 (8.08 g, 68.0%). The compound may be re-crystallized from ethanol.

### EXAMPLE 2

### (1α,2α,3α,4α,5α,6α)-5-hydroxy-6-(4,4′-dimethoxytrityloxy)-7-oxabicyclo[2.2.1]heptane-2,3-dicarboxylic acid (3)

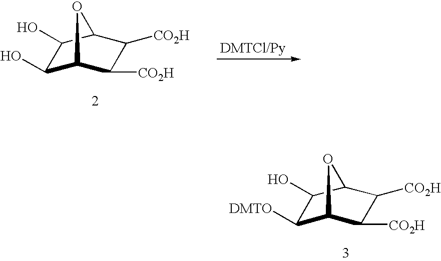

4,4′-Dimethoxytrityl chloride (7.75 g, 22.9 mmol) was added in four portions to a solution of compound 2 (3.39 g, 15.5 mmol) in pyridine (50 mL) over a period of 2 days. The solvent was evaporated, and the residue was treated with ethyl acetate (200 mL) and 1 M aqueous triethylammonium acetate (20 mL). The organic solution was washed with 1 M aqueous triethylammonium acetate (20 mL), treated with MeOH (20 mL), dried over Na₂SO₄, and evaporated. The residue was dissolved in ethyl acetate (50 mL) and treated with ether (50 mL). A crystalline precipitate was collected, washed with ether, and dried to give 3 (6.28 g, 65%).

### EXAMPLE 3

### (1α,2α,3α,4α,5α,6α)-5-(acetoxy)-6-(4,4′-dimethoxytrityloxy)-7-oxabicyclo[2.2.1]-heptane-2,3-dicarbanhydride (4)

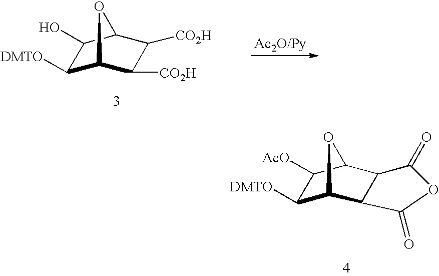

Compound 3 (1.57 g, 3.0 mmol) was treated with acetic anhydride (3.0 g) and pyridine (15 mL) for 3 h at room temperature. The mixture was evaporated and co-evaporated with pyridine (5×15 mL) to give the title compound as a colorless foam, which was used in the next step without any further purification.

### EXAMPLE 4

Support Medium 5

Aminoalkyl controlled pore glass (4.0 g, 0.51 mmol) was gently shaken with compound 4 (2.65 mmol) in pyridine (17 mL) overnight. The suspension was filtered, and the solid support was washed with pyridine (3×20 mL). The collected solution was evaporated, the residue was, upon treatment with acetic anhydride as described above, stored for loading another portion of aminoalkyl CPG. The solid support was additionally washed with ethyl acetate, dried, and capped by treating with a mixture of Ac₂O/pyridine/N-methylimidazole/THF (10:10:10:70) for 3 h at room temperature. Finally, the solid support 5 was washed with MeCN and ethyl acetate and dried. The loading of 5 (57±0.4 μmol g⁻¹) was determined by the standard DMT assay.

### EXAMPLE 5

Support Medium 6

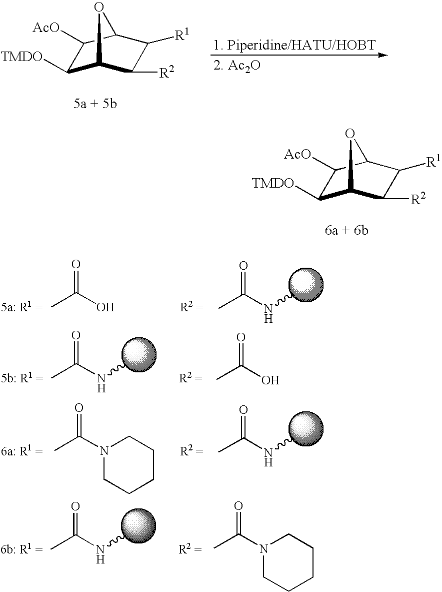

The solid support 5 (1.0 g) was treated with 0.4 M HATU and 0.3 M HOBT in MeCN-pyridine (4:1, 6 mL) for 5 minutes. The liquid phase was removed, and the solid support was treated with 0.5 M piperidine in MeCN (5 mL) for 15 minutes. The solid support was washed with MeCN (5×10 mL) and capped with a mixture of Ac₂O/pyridine/N-methylimidazole/THF (10:10:10:70) for 3 hours at room temperature. Finally, the solid support 5 was washed with MeCN and ethyl acetate and dried. The loading of 5 (57±0.4 μmol g⁻¹) was determined by the standard DMT assay.

### EXAMPLE 6

Support Medium 7

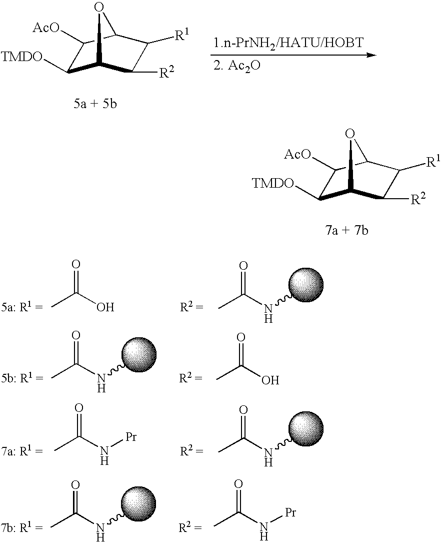

The solid support 5 (1.0 g) was treated with 0.4 M HATU and 0.3 M HOBT in MeCN-pyridine (4:1, 6 mL) for 5 minutes. The liquid phase was removed, and the solid support was treated with 0.5 M n-propylamine in MeCN (5 mL) for 15 minutes. The solid support was washed with MeCN (5×10 mL) and capped with a mixture of Ac₂O/pyridine/N-methylimidazole/THF (10:10:10:70) for 3 hours at room temperature. Finally, the solid support 5 was washed with MeCN and ethyl acetate and dried. The loading of 5 (57±0.4 μmol g⁻¹) was determined by the standard DMT assay.

### EXAMPLE 7

Support Medium 8

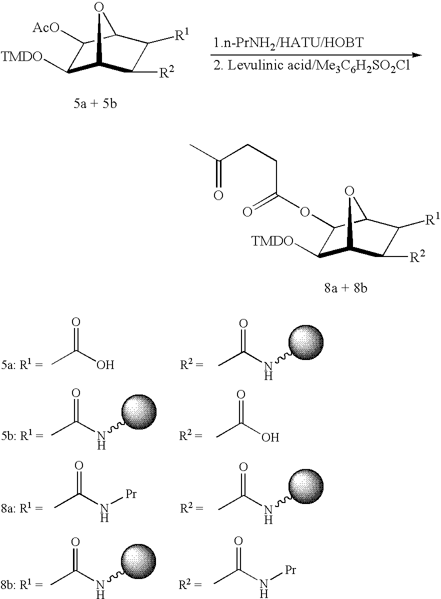

The solid support 5 (1.0 g) is treated with 0.4 M HATU and 0.3 M HOBT in MeCN-pyridine (4:1, 6 mL) for 5 minutes. The liquid phase is removed, and the solid support is treated with 0.5 M n-propylamine in MeCN (2×5 mL) for 15 and 90 minutes. The solid support is washed with MeCN (5×10 mL) and capped with a mixture of 0.5 M levulinic acid, 0.5 M mesitylene sulfonyl chloride, 0.5 M N-methylimidazole, and 1.5 M ethyldiisopropylamine in pyridine/THF (25:75) for 6 hours at room temperature. Finally, the solid support 8 is washed with MeCN and ethyl acetate and dried. The loading of 8 (57±0.4 μmol g⁻¹) was determined by the standard DMT assay.

### EXAMPLE 8

Synthesis of Oligonucleotides on Support Media 5-7

The oligonucleotide synthesis was performed on an ABI 380B DNA Synthesizer on a 1 to 4 μmol scale according to the manufacturer's recommendations. The standard and 2′-O-(2-methoxyethyl) phosphoramidites were used as 0.1 M solutions in anhydrous MeCN. The oxidation step was carried out with the standard iodine reagent or with t-butyl hydroperoxide (10% in MeCN) for 10 minutes. The preparation of oligonucleotide phosphorothioates was carried out using 3H-1,2-benzodithiol-3-one 1,1-dioxide (0.05 M in MeCN) as a sulfur-transfer reagent. Optionally, oligonucleotide phosphorothioates were synthesized using oxidation with the standard iodine reagent or t-butyl hydroperoxide solution for the linkage between the solid support and the 3′-terminal nucleoside while the internucleosidic linkages were sulfurized in a conventional manner.

The detritylation time for the solid supports 5 and 6 was extended to 6 minutes while the solid support 7 was detritylated according to the standard protocol. The coupling time of 10 minutes was used for 2′-O-(2-methoxyethyl) phosphoramidites and for the attachment of the 3′-terminal nucleoside residues to universal solid supports 5-7.

### EXAMPLE 9

Analysis of Product Distribution in Oligonucleotides Synthesized on Support Media 5-7

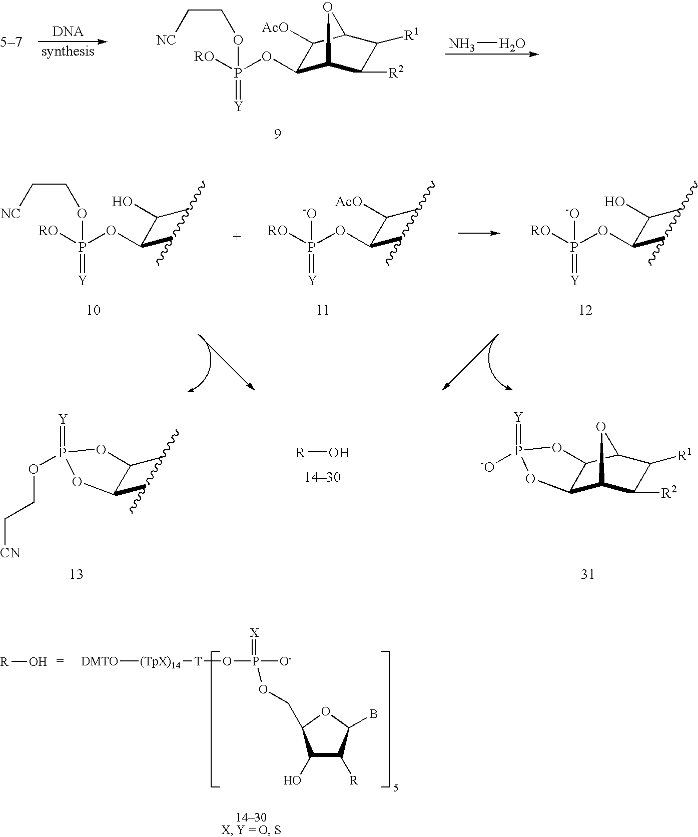

The solid support-bound oligonucleotides 9 were deprotected with concentrated aqueous ammonium hydroxide under the standard conditions (8 h at 55° C.). The liquid phase was withdrawn and evaporated to give crude oligonucleotides 14-30 specified in Table 1. Spectrophotometric determination at 260 nm demonstrated the crude yields being in the expected range of 70 to 100 OD μmol⁻¹ (Table 2). The 5′-DMT protected oligonucleotides were analyzed by reverse phase HPLC on a DeltaPak C18 column (Waters, 3.8×300 mm) using a linear gradient from 0 to 50% MeCN in 0.1 M ammonium acetate. The crude products were further characterized by ES MS to show no side products bearing a 3′-derivatized hydroxy group.

### EXAMPLE 10

Experimental Setup for Recording the Kinetics of Chemical Reactions on Solid Surfaces in Continuous Flow

An apparatus comprising a liquid metering pump, thermostat, reagent loop, switching valve, reaction vessel, and data acquisition system was assembled. A reagent was continuously delivered to the reagent loop where it was brought to a desired temperature. On leaving the loop, the reagent contacted a solid phase placed in the thermostated plug-flow reactor. The products dissolved in the reagent were eluted to a detector chosen in accordance with the nature of compounds whose concentration was to be measured. The data were acquired and stored by an attached computer system. Optionally, fractions of the reagent containing dissolved products were collected and re-analyzed off-line by a different method, for instance, HPLC.

### EXAMPLE 11

Determination of the Time Required for the 95% Release of Oligonucleotides Synthesized on 5-7

A solid-support-bound oligonucleotide 9 (0.1 to 1 μmol) was placed in the reaction vessel and thermostated at 27.05° C. for 30 minutes. The data collection was started, and aqueous ammonium hydroxide (14.3 M, 27.1%) was delivered to the reaction vessel for 7 to 10 hours at a constant flow of 0.5 mL min⁻¹. The recorded data were integrated and deconvoluted to determine the time required for the 95% release of oligonucleotides 14-30 from the universal solid support 7 (Table 1). The oligonucleotide 17 synthesized on solid supports 5 and 6 was released at a rate equal to that for the solid support 7.

TABLE 1

| Oligonucleotide | | | | | 95% release |
|---|---|---|---|---|---|
| Compound | Base | R | X | Y | (minutes) |
| 14 | A | H | O | O | 238 |
| 15 | G | H | O | O | 272 |
| 16 | C | H | O | O | 251 |
| 17 | T | H | O | O | 256 |
| 18 | A | H | S | S | 343 |
| 19 | G | H | S | S | 456 |
| 20 | G | H | S | O | 280 |
| 21 | C | H | S | S | 365 |
| 22 | T | H | S | S | 382 |
| 23 | G | MOE | O | O | 180 |
| 24 | 5-Me-U | MOE | O | O | 129 |
| 25 | A | MOE | S | S | 161 |
| 26 | G | MOE | S | S | 212 |
| 27 | G | MOE | S | O | 185 |
| 28 | 5-Me-C | MOE | S | S | 159 |
| 29 | 5-Me-U | MOE | S | S | 182 |
| 30 | U | Ome | O | O | 123 |

Abbreviations:
A = adenine, G = guanine, C = cytosine, T = thymine
5-Me-C = 5-methyl-cytosine; 5-Me-U = 5-methyl uracil
MOE = methoxyethoxy
OMe = methoxy
H = hydrogen; O = oxygen; S = sulfur

TABLE 2

| | | | | | Crude |
|---|---|---|---|---|---|
| | | | | | Yield, OD |
| Compound | Base | R | X | Y | μmol⁻¹ |
| 14 | A | H | O | O | 84 |
| 15 | G | H | O | O | 82 |
| 16 | C | H | O | O | 101 |
| 17 | T | H | O | O | 94 |
| 25 | A | MOE | S | S | 72 |
| 26 | G | MOE | S | S | 87 |
| 27 | 5-Me-C | MOE | S | S | 110 |
| 28 | 5-Me-U | MOE | S | S | 110 |

### EXAMPLE 12

Universal Solid Support 31

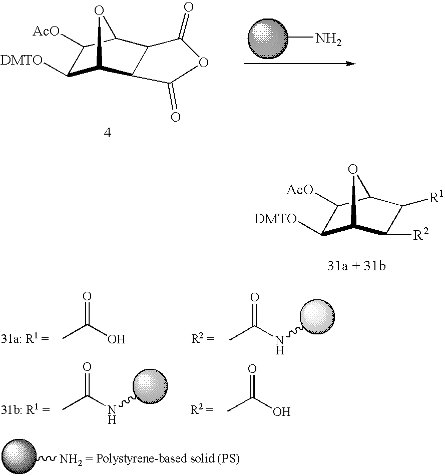

A polystyrene-based solid support Primer 30 HL (13.5 g, 2.7 mmol) was gently shaken with compound 4 (0.73 g, 1.34 mmol) in pyridine (150 mL) overnight. The suspension was filtered, and the solid support was washed with pyridine (3×100 mL). The solid support was additionally washed with ethyl acetate, dried, and capped by treating with a mixture of Ac₂O/pyridine/N-methylimidazole/THF (10:10:10:70) for 3 hours at room temperature. Finally, the solid support 31 was washed with MeCN and ethyl acetate and dried. The loading of 31 (96 μmol g⁻¹) was determined by the standard DMT assay.

### EXAMPLE 13

Universal Solid Support 32

The solid support 31 (1.0 g) was treated with 0.2 M HATU and 0.23 M HOBT in MeCN-pyridine (4:1, 10 mL) for 5 minutes. The liquid phase was removed, and the solid support was treated with 0.5 M piperidine in MeCN (10 mL) for 15 minutes. The solid support was washed with MeCN (5×10 mL) and capped with a mixture of Ac₂O/pyridine/N-methylimidazole/THF (10:10:10:70) for 3 hours at room temperature. Finally, the solid support 32 was washed with MeCN and ethyl acetate and dried. The loading of 32 (96 μmol g⁻¹) was determined by the standard DMT assay.

### EXAMPLE 14

Universal Solid Support 33

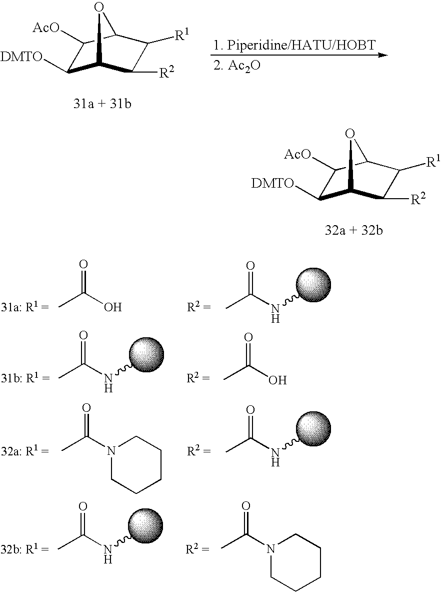

The solid support 31 (10.0 g) was treated with 0.2 M HATU and 0.2 M HOBT in MeCN-pyridine (4:1, 80 mL) for 5 minutes. The liquid phase was removed, and the solid support was treated with 0.5 M n-propylamine in MeCN (100 mL) for 15 minutes. The solid support was washed with MeCN (5×100 mL) and capped with a mixture of Ac₂O/pyridine/N-methylimidazole/THF (10:10:10:70) for 3 h at room temperature. Finally, the solid support 33 was washed with MeCN and ethyl acetate and dried. The loading of 33 (96 μmol g⁻¹) was determined by the standard DMT assay.

### EXAMPLE 15

Universal Solid Support 34

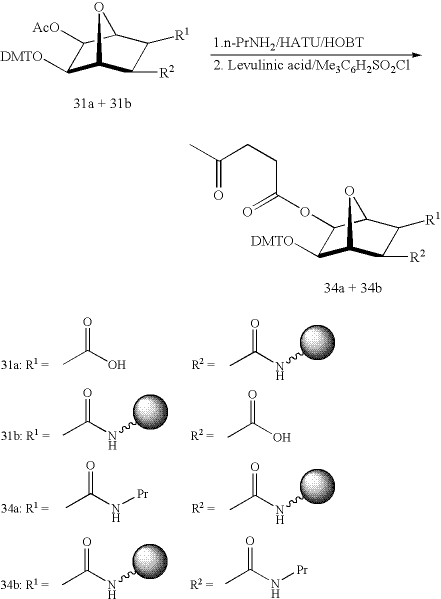

The solid support 31 (1.0 g) is treated with 0.2 M HATU and 0.2 M HOBT in MeCN-pyridine (4:1, 10 mL) for 5 minutes. The liquid phase is removed, and the solid support is treated with 0.5 M n-propylamine in MeCN (2×5 mL) for 15 and 90 minutes. The solid support is washed with MeCN (5×10 mL) and capped with a mixture of 0.5 M levulinic acid, 0.5 M mesitylene sulfonyl chloride, 0.5 M N-methylimidazole, and 1.5 M ethyldiisopropylamine in pyridine/THF (25:75) for 6 h at room temperature. Finally, the solid support 34 is washed with MeCN and ethyl acetate and dried. The loading of 34 (96 μmol g⁻¹) is determined by the standard DMT assay.

### EXAMPLE 16

Universal Solid Support 35

An aminopolystyrene PS 200 (10 g, 2 mmol) was gently shaken with compound 4 (0.73 g, 1.34 mmol) in pyridine (150 mL) overnight. The suspension was filtered, and the solid support was washed with pyridine (3×100 mL). The solid support was additionally washed with ethyl acetate, dried, and capped by treating with a mixture of Ac₂O/pyridine/N-methylimidazole/THF (10:10:10:70) for 3 h at room temperature. Finally, the solid support 35 was washed with MeCN and ethyl acetate and dried. The loading of 35 (ca 100 μmol g⁻¹) was determined by the standard DMT assay.

### EXAMPLE 17

Universal Solid Support 36

The solid support 35 (10.0 g) was treated with 0.2 M HATU and 0.2 M HOBT in MeCN-pyridine (4:1, 80 mL) for 5 minutes. The liquid phase was removed, and the solid support was treated with 0.5 M n-propylamine in MeCN (100 mL) for 15 minutes. The solid support was washed with MeCN (5×100 mL) and capped with a mixture of Ac₂O/pyridine/N-methylimidazole/THF (10:10:10:70) for 3 hours at room temperature. Finally, the solid support 36 was washed with MeCN and ethyl acetate and dried. The loading of 36 (ca. 100 μmol g¹) was determined by the standard DMT assay.

### EXAMPLE 18

Universal Solid Support 37

A Merckogel solid support (2 mmol) was gently shaken with compound 4 (0.73 g, 1.34 mmol) in pyridine (150 mL) overnight. The suspension was filtered, and the solid support was washed with pyridine (3×100 mL). The solid support was additionally washed with ethyl acetate, dried, and capped by treating with a mixture of Ac₂O/pyridine/N-methylimidazole/THF (10:10:10:70) for 3 hours at room temperature. Finally, the solid support 37 was washed with MeCN and ethyl acetate and dried. The loading of 37 (ca 100 μmol g⁻¹) was determined by the standard DMT assay.

### EXAMPLE 19

Universal Solid Support 38

The solid support 37 (10.0 g) was treated with 0.2 M HATU and 0.2 M HOBT in MeCN-pyridine (4:1, 80 mL) for 5 minutes. The liquid phase was removed, and the solid support was treated with 0.5 M n-propylamine in MeCN (100 mL) for 15 minutes. The solid support was washed with MeCN (5×100 mL) and capped with a mixture of Ac₂O/pyridine/N-methylimidazole/THF (10:10:10:70) for 3 hours at room temperature. Finally, the solid support 38 was washed with MeCN and ethyl acetate and dried. The loading of 38 (ca. 100 μmol g⁻¹) was determined by the standard DMT assay.

### EXAMPLE 20

Universal Solid Support 39

A Tentagel solid support (2 mmol) was gently shaken with compound 4 (0.73 g, 1.34 mmol) in pyridine (150 mL) overnight. The suspension was filtered, and the solid support was washed with pyridine (3×100 mL). The solid support was additionally washed with ethyl acetate, dried, and capped by treating with a mixture of Ac₂O/pyridine/N-methylimidazole/THF (10:10:10:70) for 3 hours at room temperature. Finally, the solid support 39 was washed with MeCN and ethyl acetate and dried. The loading of 39 (ca 100 μmol g⁻¹) was determined by the standard DMT assay.

### EXAMPLE 21

Universal Solid Support 40

The solid support 39 (10.0 g) was treated with 0.2 M HATU and 0.2 M HOBT in MeCN-pyridine (4:1, 80 mL) for 5 minutes. The liquid phase was removed, and the solid support was treated with 0.5 M n-propylamine in MeCN (100 mL) for 15 minutes. The solid support was washed with MeCN (5×100 mL) and capped with a mixture of Ac₂O/pyridine/N-methylimidazole/THF (10:10:10:70) for 3 hours at room temperature. Finally, the solid support 40 was washed with MeCN and ethyl acetate and dried. The loading of 40 (ca. 100 μmol g⁻¹) was determined by the standard DMT assay.

### EXAMPLE 22

Utilization of —OH Containing Supports

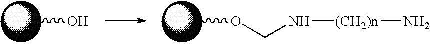

The hydroxyl containing support is treated with CDI followed by 1,6-diaminohexane. This provides amine-containing supports.

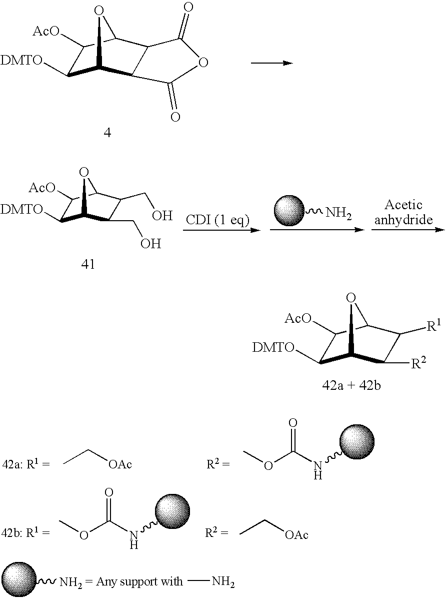

### EXAMPLE 23

Carbamate Linked Supports

Compound 4 is reduced to the diol compound 41 using LiAlH₄. It is treated with 1 equivalent of CDI followed by the reaction support followed by capping with acetic anhydride. This results in carbamate containing reaction supports.

### EXAMPLE 24

Bis-hydroxylation of 5-Norbornene-2-carboxylic acid

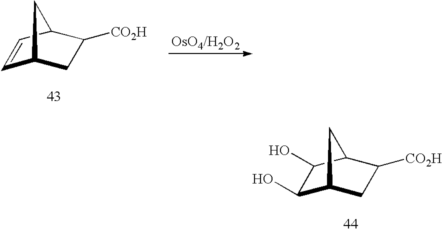

A solution of commercial 5-norbomene-2-carboxylic acid, (43) (7.53 g, 54.5 mmol) in hydrogen peroxide (30% aqueous, 2.97 g, 87.2 mmol), acetone (72.5 mL), ether (18.1 mL), and t-butanol (6.2 mL) is treated with osmium tetroxide (56 mg, 0.22 mmol) in t-butanol (2.86 mL) for 4 days at 28-30° C. The reaction mixture is treated with ether (90 mL) and kept at 4° C. for 1 h. The precipitate is filtered off, washed with ether and dried to give pure 44.

### EXAMPLE 25

Dimethoxytritylation of Compound 44

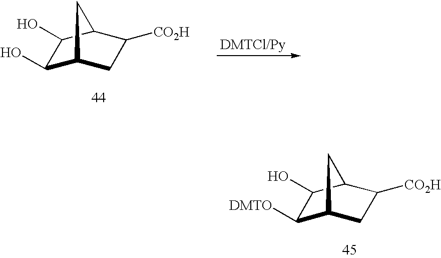

4,4′-Dimethoxytrityl chloride (7.75 g, 22.9 mmol) is added in four portions to a solution of compound 44 (2.67 g, 15.5 mmol) in pyridine (50 mL) over a period of 2 days. The solvent is evaporated, and the residue is treated with ethyl acetate (200 mL) and 1 M aqueous triethylammonium acetate (20 mL). The organic solution is washed with 1 M aqueous triethylammonium acetate (20 mL), treated with MeOH (20 mL), dried over Na₂SO₄, and evaporated. The residue is dissolved in ethyl acetate (50 mL) and treated with ether (50 mL). A crystalline precipitate is collected, washed with ether, and dried to give 45.

### EXAMPLE 26

Blocking of Hydroxyl Group as Acetate (46)

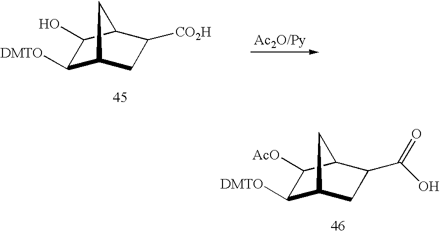

Compound 45 (3.0 mmol) is treated with acetic anhydride (3.0 g) and pyridine (15 mL) for 3 h at room temperature. The mixture is evaporated and co-evaporated with pyridine (5×15 mL) to give the title compound as a colorless foam, which is used in the next step without any further purification.

### EXAMPLE 27

Loading of Linker to CPG Solid Support

The carboxylic acid (1.0 equivalents), aminoalky controlled pore glass (8 g; 143 μM/g; 1 equivalent); Hüinig's base (0.59 g; 0.8 ml; 4 equivalents) and HBTU (1.09 g; 2.5 equivalents) are taken up in a 250 mL round bottomed flask and anhydrous acetonitrile (100 mL) is added. The flask is shaken at room temperature using a mechanical shaker for 6 hours. The solid support is then filtered using a coarse sintered funnel, washed with DMF (100 mL), acetonitrile (100 mL), methanol (100 mL) and then with ether (300 mL). The dried support is evaluated for loading using the standard DMT assay conditions and then capped with acetic anhydride in the standard manner.

### EXAMPLE 28

Synthesis of Fully-Modified Phosphorothioate 20-mer

| | |
|---|---|
| 5′-d(TCC-CGC-CTG-TGA-CAT-GCA-TT)-3′ | (SEQ ID NO: 1) |

Synthesis of above sequence is performed on an ABI 390Z DNA/RNA Synthesizer on a 15 μM scale using cyanoethyl phosphoramidites and the above prepared CPG solid support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner to afford the desired oligonucleotide.

### EXAMPLE 29

Synthesis of Fully-Modified Phosphorothioate 19-mer

| | |
|---|---|
| 5′-d(GTT-CTC-GCT-GGT-GAG-TTT-C)-3′ | (SEQ ID NO: 2) |

Synthesis of above sequence is performed on an ABI 390Z DNA/RNA Synthesizer on a 15 μM scale using cyanoethyl phosphoramidites and the above prepared CPG solid support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner to afford the desired phosphorothioate oligonucleotide.

### EXAMPLE 30

Loading of Linker to Amino-Derivatized HL30 Solid Support

Carboxylic acid (1.0 equivalents), Pharmacia's amino derivatized Primer support (8 g; 143 micromole/g; 1 equivalent); Hünig's base (0.59 g; 0.8 ml; 4 equivalents) and HBTU (1.09 g; 2.5 equivalents) are taken up in a 250 ml round bottomed flask and anhydrous acetonitrile (100 mL) is added. The flask is shaken at room temperature using a mechanical shaker for 6 hours. The solid support is then filtered using a coarse sintered funnel, washed with DMF (100 mL), acetonitrile (100 mL), methanol (100 mL) and then with ether (300 mL). The dried support is evaluated for loading using the standard DMT assay conditions and then capped with acetic anhydride in the standard manner.

### EXAMPLE 31

Synthesis of Fully-Modified Phosphorothioate 20-mer

| | |
|---|---|
| 5′-d(TCC-CGC-CTG-TGA-CAT-GCA-TT)-3′ | (SEQ ID NO: 1) |

Synthesis of above sequence is performed on a Pharmacia OligoPilot II Synthesizer on a 135 μM scale using cyanoethyl phosphoramidites and the above prepared Primer support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner to afford the purified desired oligonucleotide.

### EXAMPLE 32

Synthesis of Fully-Modified Phosphorothioate 19-mer

| | |
|---|---|
| 5′-d(GTT-CTC-GCT-GGT-GAG-TTT-C)-3′ | (SEQ ID NO: 2) |

Synthesis of above sequence is performed on a Pharmacia OligoPilot II Synthesizer on a 323 μM scale using cyanoethyl phosphoramidites and the above prepared Primer support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner to afford the phosphorothioate oligonucleotide.

### EXAMPLE 33

Synthesis of Fully-Modified Phosphorothioate 19-mer

| | |
|---|---|
| 5′-d(CTA-CGC-TTT-CCA-CGC-ACA-G)-3′ | (SEQ ID NO: 3) |

Synthesis of above sequence is performed on a Pharmacia OligoPilot II Synthesizer on a 323 μM scale using cyanoethyl phosphoramidites and the above prepared Primer support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner to afford the phosphorothioate oligonucleotide.

### EXAMPLE 34

Synthesis of Fully-Modified Phosphorothioate 20-mer

| | |
|---|---|
| 5′-d(TCC-CGC-CTG-TGA)-2′-methoxyethyl-(CAT-GCA-TT)-3′ | (SEQ ID NO: 4) |

Synthesis of above sequence is performed on an OligoPilot II DNA/RNA Synthesizer on a 282 μM scale using cyanoethyl phosphoramidites and derivatized primer support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner.

### EXAMPLE 35

Synthesis of Fully-Modified Phosphorothioate 20-mer

| | |
|---|---|
| 5′-d(TCC-CGC-CTG-TGA)-2′-methoxyethyl-(CAT-GCA-TT)-3′ | (SEQ ID NO: 4) |

Synthesis of above sequence is performed on a Pharmacia OligoPilot II Synthesizer at 250 μM scale using cyanoethyl phosphoramidites and derivatized primer support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner.

### EXAMPLE 36

Bis-hydroxylation of 7-Oxabicyclo[2.2.1 ]hept-5-ene-2-carboxylic acid

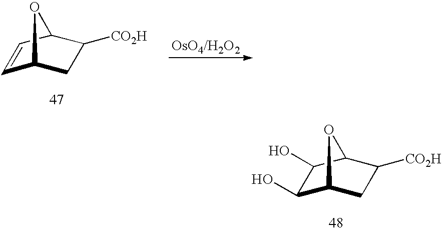

A solution of commercial 7-Oxabicyclo[2.2.1 ]hept-5-ene-2-carboxylic acid, (54.5 mmol) in hydrogen peroxide (30% aqueous, 2.97 g, 87.2 mmol), acetone (72.5 mL), ether (18.1 mL), and t-butanol (6.2 mL) is treated with osmium tetroxide (56 mg, 0.22 mmol) in t-butanol (2.86 mL) for 4 days at 28-30° C. The reaction mixture is treated with ether (90 mL) and kept at 4° C. for 1 h. The precipitate is filtered off, washed with ether and dried to give pure compound.

### EXAMPLE 37

Dimethoxytritylation of bis-Hydroxy Compound

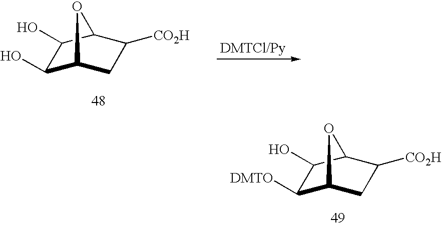

4,4′-Dimethoxytrityl chloride (7.75 g, 22.9 mmol) is added in four portions to a solution of compound 2 (2.67 g, 15.5 mmol) in pyridine (50 mL) over a period of 2 days. The solvent is evaporated, and the residue is treated with ethyl acetate (200 mL) and 1 M aqueous triethylammonium acetate (20 mL). The organic solution is washed with 1 M aqueous triethylammonium acetate (20 mL), treated with MeOH (20 mL), dried over Na₂SO₄, and evaporated. The residue is dissolved in ethyl acetate (50 mL) and treated with ether (50 mL). A crystalline precipitate is collected, washed with ether, and dried to give 49.

### EXAMPLE 38

Blocking of Hydroxyl Group as Acetate

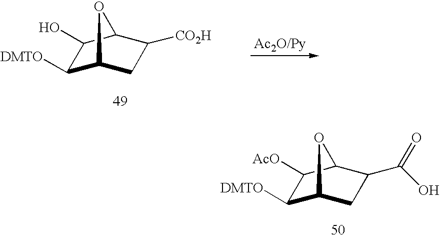

Compound 49 (3.0 mmol) is treated with acetic anhydride (3.0 g) and pyridine (15 mL) for 3 h at room temperature. The mixture is evaporated and co-evaporated with pyridine (5×15 mL) to give the title compound as a colorless foam, which is used in the next step without any further purification.

### EXAMPLE 39

Loading of Linker to CPG Solid Support

The carboxylic acid (8) (1.0 equivalents), aminoalky controlled pore glass (8 g; 143 micromole/g; 1 equivalent); Hünig's base (0.59 g; 0.8 ml; 4 equivalents) and HBTU (1.09 g; 2.5 equivalents) are taken up in a 250 ml round bottomed flask and anhydrous acetonitrile (100 ml) is added. The flask is shaken at room temperature using a mechanical shaker for 6 hours. The solid support is then filtered using a coarse sintered funnel, washed with DMF (100 ml), acetonitrile (100 ml), methanol (100 ml) and then with ether (300 ml). The dried support is evaluated for loading using the standard DMT assay conditions and then capped with acetic anhydride in the standard manner.

### EXAMPLE 40

Synthesis of Fully-Modified Phosphorothioate 20-mer

| | |
|---|---|
| 5′-d(TCC-CGC-CTG-TGA-CAT-GCA-TT)-3′ | (SEQ ID NO: 1) |

Synthesis of above sequence is performed on an ABI 390Z DNA/RNA Synthesizer on a 15 micromole scale using cyanoethyl phosphoramidites and the above prepared CPG solid support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner to afford the desired oligonucleotide.

### EXAMPLE 41

Synthesis of Fully-Modified Phosphorothioate 19-mer

| | |
|---|---|
| 5′-d(GTT-CTC-GCT-GGT-GAG-TTT-C)-3′ | (SEQ ID NO: 2) |

Synthesis of above sequence is performed on an ABI 390Z DNA/RNA Synthesizer on a 15 μM scale using cyanoethyl phosphoramidites and the above prepared CPG solid support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner to afford the desired phosphorothioate oligonucleotide.

### EXAMPLE 42

Loading of Linker to Amino-Derivatized HL30 Solid Support

The carboxylic acid (1.0 equivalents), Pharmacia's amino derivatized Primer support (8 g; 143 micromole/g; 1 equivalent); Hünig's base (0.59 g; 0.8 ml; 4 equivalents) and HBTU (1.09 g; 2.5 equivalents) are taken up in a 250 mL round bottomed flask and anhydrous acetonitrile (100 mL) is added. The flask is shaken at room temperature using a mechanical shaker for 6 hours. The solid support is then filtered using a coarse sintered funnel, washed with DMF (100 mL), acetonitrile (100 mL), methanol (100 mL) and then with ether (300 mL). The dried support is evaluated for loading using the standard DMT assay conditions and then capped with acetic anhydride in the standard manner.

### EXAMPLE 43

Synthesis of Fully-Modified Phosphorothioate 20-mer

| | |
|---|---|
| 5′-d(TCC-CGC-CTG-TGA-CAT-GCA-TT)-3′ | (SEQ ID NO: 1) |

Synthesis of above sequence is performed on a Pharmacia OligoPilot II Synthesizer on a 135 μM scale using cyanoethyl phosphoramidites and the above prepared Primer support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner to afford the purified desired oligonucleotide.

### EXAMPLE 44

Synthesis of Fully-Modified Phosphorothioate 19-mer

| | |
|---|---|
| 5′-d(GTT-CTC-GCT-GGT-GAG-TTT-C)-3′ | (SEQ ID NO: 2) |

Synthesis of above sequence is performed on a Pharmacia OligoPilot II Synthesizer on a 323 μM scale using cyanoethyl phosphoramidites and the above prepared Primer support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner to afford the phosphorothioate oligonucleotide.

### EXAMPLE 45

Synthesis of Fully-Modified Phosphorothioate 19-mer

| | |
|---|---|
| 5′-d(CTA-CGC-TTT-CCA-CGC-ACA-G)-3′ | (SEQ ID NO: 3) |

Synthesis of above sequence is performed on a Pharmacia OligoPilot II Synthesizer on a 323 μM scale using cyanoethyl phosphoramidites and the above prepared Primer support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner to afford the phosphorothioate oligonucleotide.

### EXAMPLE 46

Synthesis of Fully-Modified Phosphorothioate 20-mer

| | |
|---|---|
| 5′-d(TCC-CGC-CTG-TGA)-2′-methoxyethyl-(CAT-GCA-TT)-3′ | (SEQ ID NO: 4) |

Synthesis of above sequence is performed on an OligoPilot II DNA/RNA Synthesizer on a 282 μM scale using cyanoethyl phosphoramidites and derivatized primar support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner.

### EXAMPLE 47

Synthesis of Fully-Modified Phosphorothioate 20-mer

| | |
|---|---|
| 5′-d(TCC-CGC-CTG-TGA)-2′-methoxyethyl-(CAT-GCA-TT)-3′ | (SEQ ID NO: 4) |

Synthesis of above sequence is performed on a Pharmacia OligoPilot II Synthesizer at 250 μM scale using cyanoethyl phosphoramidites and derivatized primar support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner.

### EXAMPLE 48

Synthesis of the Catalyst Solution

Purification of t-Butyl Alcohol:

tert-Butyl alcohol was shaken for 48 h with solid potassium permanganate, filtered, dried with magnesium sulfate, and distilled from fresh from solid potassium permanganate directly into the glass stoppered bottle used for Synthesis of osmium tetroxide catalyst solution. (Note: Unless impurities are removed by this procedure, the catalyst solution was unstable; it turned black and was not suitable for the hydroxylation reactions below).

Osmium Tetroxide Catalyst Solution:

The content of a 1 g sealed vial of osmium tetroxide was dissolved in 200 mL of purified t-butyl alcohol. The pale green solution was treated with 3 drops of 30% hydrogen peroxide and allowed to remain at room temperature for 1 day. If the solution became dark, the dropwise addition of 30% hydrogen peroxide was repeated until the pale green color persisted. This solution is stable for at least one year at room temperature. Each mL contains 2×10−5 mole of osmium tetroxide.

### EXAMPLE 49

Synthesis of Compound 2, Hydroxylation Reaction

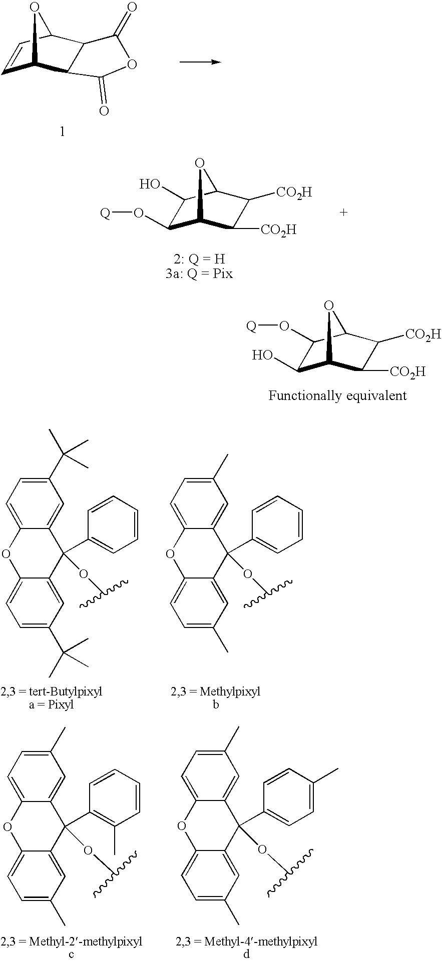

To a stirred solution of exo-3,6-epoxy-1,2,3,6-tetrahydrophthalic anhydride (1, 1 Kg; 6 moles) (Note: takes overnight stirring to dissolve) in 8 L of acetone were added 1 L of osmium tetroxide catalyst solution (Example 48, 1 hour addition), 1 L of 30% hydrogen peroxide (800 mmole) (1 hour addition), followed by 2 L of ether (1 hour addition). The reaction mixture became brown and the solution was stirred mechanically and maintained at 30° C. for 24 h (Note: internal temperature should not go above 45° C.). At that time a white solid was formed. An additional 10 L of ether was added (1 hour addition) and the suspension was cooled in an ice bath for 5 to 6 hours. The solid obtained was filtered and washed with ether (3 L) and dried in air.

This crude product was treated with 15 L of warm methanol and filtered to remove 50 g of a white solid. The methanolic filtrate was concentrated to 10 L and absolute ether was added to precipitate the product. The precipitate was recrystallized from methanol-isoamyl acetate (alternatively 50% aqueous dioxane can be used). The crystalline material (2) was dried overnight using reduced pressure at 80° C. over phosphorous pentoxide (mp 199-200° dec).

### EXAMPLE 50

Synthesis of Pixyl Derivative Compound (3a)

9-(2,3-tert-Butyl)xanthen-9-yl chloride was added slowly in portions (2.5, 0.25, 0.25, 0.1, 0.08 mole portions) to a solution of the diol, compound 2 in pyridine. The final reaction mixture contained mono-pixyl, bis-pixyl, and the starting diol in a ratio of 90:6:4 (Note: ¹H NMR in pyridine-d). Triethylamine (500 mL) was added, the reaction mixture was evaporated and co-evaporated with toluene (3×2 L). The residue was dissolved in water (10 L), ethyl acetate (9 L), and triethylamine (300 mL). The aqueous phase was separated, and the organic phase was washed with water (4×2.5 L). At the end of washing, precipitation from aqueous phase started. Both the organic and the combined aqueous phase were left separately overnight at room temperature. The precipitates were formed in both phases. These were filtered off. The organic phase was diluted with half-volume of ether and left at room temperature. The additional solid material was collected by filtration after two days and after about a week. Total yield 946 g (65%) of Compound 3a. All solids were desired product and obtained as free acid in a comparable purity. All the solid material obtained were combined and stirred in ethyl acetate (10 L) overnight, filtered, dried in vacuum to give the product as free acid. If needed, the material was dissolved in anhydrous pyridine and concentrated to remove traces of moisture.

### EXAMPLE 51

Synthesis of Compound (4a)

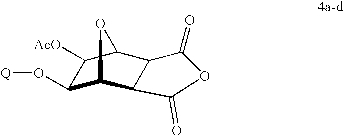

Compound 3a (157 g) was dissolved in anhydrous pyridine (1500 mL) and treated with acetic anhydride (300 g) overnight at room temperature. The mixture was evaporated and co-evaporated with toluene (5×1500 mL) to give the title compound (4a) as a pale brown to colorless solid.

### EXAMPLE 52

Synthesis of Pixyl Derivative Compound (3b)

9-(2,3-Dimethyl)xanthen-9-yl chloride was added slowly in portion wise (2.5, 0.25, 0.25, 0.1, 0.08 mole portions) to a solution of the diol, Compound 2 in pyridine. The final reaction mixture contained mono-pixyl, bis-pixyl, and the starting diol in a ratio of 90:6:4 (Note: ¹H NMR in pyridine-d). Triethylamine (500 mL) was added, the reaction mixture was evaporated and co-evaporated with toluene (3×2 L). The residue was dissolved in water (10 L), ethyl acetate (9 L), and triethylamine (300 mL). The aqueous phase was separated, and the organic phase was washed with water (4×2.5 L). At the end of washing, precipitation from aqueous phase started. Both the organic and the combined aqueous phase were left separately overnight at room temperature. The precipitates were formed in both phases. These were filtered off. The organic phase was diluted with half-volume of ether and left at room temperature. The additional solid material was collected by filtration after two days and after about a week. Total yield=55% Compound 3b. All solids were desired product and obtained as free acid in a comparable purity. All the solid material obtained were combined and stirred in ethyl acetate (10 L) overnight, filtered, dried in vacuum to give the product as free acid. If needed, the material was dissolved in anhydrous pyridine and concentrated to remove traces of moisture.

### EXAMPLE 53

Synthesis of Compound (4b)

Compound 3b (157 g) was dissolved in anhydrous pyridine (1500 mL) and treated with acetic anhydride (300 g) overnight at room temperature. The mixture was evaporated and co-evaporated with toluene (5×1500 mL) to give the title compound as a pale brown to colorless solid.

### EXAMPLE 54

Synthesis of Pixyl Derivative Compound (3c)

9-(2,3-Di-tert-Butyl-2′-methyl)xanthen-9-yl chloride was added slowly in portion wise (2.5, 0.25, 0.25, 0.1, 0.08 mole portions) to a solution of the diol 2 in pyridine. The final reaction mixture contained mono-pixyl, bis-pixyl, and the starting diol in a ratio of 90:6:4 (Note: ¹H NMR in pyridine-d). Triethylamine (500 mL) was added, the reaction mixture was evaporated and co-evaporated with toluene (3×2 L). The residue was dissolved in water (10 L), ethyl acetate (9 L), and triethylamine (300 mL). The aqueous phase was separated, and the organic phase was washed with water (4×2.5 L). At the end of washing, precipitation from aqueous phase started. Both the organic and the combined aqueous phase were left separately overnight at room temperature. The precipitates were formed in both phases. These were filtered off. The organic phase was diluted with half-volume of ether and left at room temperature. The additional solid material was collected by filtration after two days and after about a week. Total yield=72%. All solids were desired product and obtained as free acid in a comparable purity. All the solid material obtained were combined and stirred in ethyl acetate (10 L) overnight, filtered, dried in vacuum to give the product as free acid. If needed, the material was dissolved in anhydrous pyridine and concentrated to remove traces of moisture.

### EXAMPLE 55

Synthesis of Compound (4c)

Compound 3c (157 g) was dissolved in anhydrous pyridine (1500 mL) and treated with acetic anhydride (300 g) overnight at room temperature. The mixture was evaporated and co-evaporated with toluene (5×1500 mL) to give the title compound as a pale brown to colorless solid.

### EXAMPLE 56

General Procedure for Synthesis of Pixyl Analog (as the Alcohols)

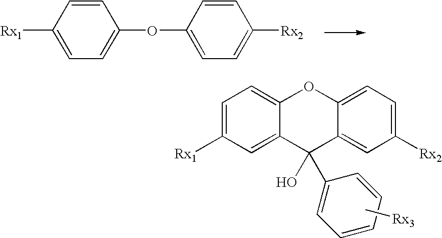

To a stirred mixture of substituted or unsubstituted diphenylether (1.01 mole), ring substituted or unsubstituted benzoic acid (1.13 mole) and anhydrous zinc chloride (400 g; 2.94 mole) is added phosphorousoxy trichloride (300 mL; 3.27 mole) slowly using an addition funnel. The reaction mixture is then slowly heated to 95° C. after the reaction starts and is monitored by tlc. After the reaction is complete, ethyl acetate (500 mL) is added, followed by water (200 mL) slowly. An additional amount of water (2500 mL) is added at a faster rate. The mixture is stirred overnight at room temperature and a solid will come out of the solution. The solid is filtered and recrystallized from methanol to afford the substituted pixyl alcohol product.

### EXAMPLE 57

Conversion of the Pixyl Analogs from Alcohols to Reactive Alkyl Halides

To a stirred solution of the substituted pixyl alcohol (Example 56, 0.982 mole) in dichloromethane (1000 mL) is added thionyl chloride (102 ml; 1.1 mole) slowly with cooling. The reaction is monitored by thin layer chromatography. When complete, the reaction is concentrated, toluene added followed by hexane to afford the pixyl analog as the chloride.

### EXAMPLE 58

Loading of Linker to CPG Solid Support (Pg=Protecting Group)

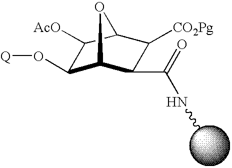

A solution of anhydride 4a (1.5 equivalents) was dissolved in pyridine (60 mL), and LCAA CPG beads (9.17 g) was added. The suspension was gently shaken for 6 h. The suspension was filtered, and the solid support was washed with pyridine (3×50 mL) and MeCN (3×50 mL). The solid support was capped by treating with a mixture of Ac₂O/pyridine/N-methylimidazole/THF (10:10:10:70) overnight at room temperature. Finally, the solid support was washed with MeCN and ethyl acetate and dried. The loading of 55±2 μmol/g was determined by the standard DMT assay.

The solid support was treated with 0.2 M HATU in MeCN-pyridine (4:1, 50 mL) for 15 min. The liquid phase was removed, and the solid support was treated with 0.5 M n-propylamine in MeCN (50 mL) for 15 min. The solid support was washed with MeCN (5×50 nL) and the treatment with HATU/n-propylamine followed by washing with MeCN (5×50 mL) was repeated. The solid support was capped with a mixture of Ac₂O/pyridine/N-methylimidazole/THF (10:10:10:70) overnight at room temperature. Finally, the solid support was washed with MeCN and ethyl acetate and dried. The loading of 40±3 μmole/g was determined by the standard DMT assay.

### EXAMPLE 59

Loading of Linker to HL30 Amino Derivatized Solid Support

A solution of anhydride 4a (1.5 equivalents) was dissolved in pyridine (60 mL), and HL30 amino derivatized solid support (10 g) was added. The suspension was gently shaken for 6 h. The suspension was filtered, and the solid support was washed with pyridine (3×50 mL) and MeCN (3×50 mL). The solid support was capped by treating with a mixture of Ac₂O/pyridine/N-methylimidazole/THF (10:10:10:70) overnight at room temperature. Finally, the solid support was washed with MeCN and ethyl acetate and dried. The loading of 100±2 μmol/g was determined by the standard DMT assay.

The solid support was treated with 0.2 M HATU in MeCN-pyridine (4:1, 50 mL) for 15 min. The liquid phase was removed, and the solid support was treated with 0.5 M n-propylamine in MeCN (50 mL) for 15 min. The solid support was washed with MeCN (5×50 mL) and the treatment with HATU/n-propylamine followed by washing with MeCN (5×50 mL) was repeated. The solid support was capped with a mixture of Ac₂O/pyridine/N-methylimidazole/THF (10:10:10:70) overnight at room temperature. Finally, the solid support was washed with MeCN and ethyl acetate and dried. The loading of 88±3 μmole/g was determined by the standard DMT assay.

### EXAMPLE 60

| | |
|---|---|
| Synthesis of fully-modified 5′-d(TCC-CGC-CTG-TGA-CAT-GCA-TT)-3′ | (SEQ ID NO: 1) |
| phosphorothioate 20-mer | |

Synthesis of above sequence is performed on an ABI 390Z DNA/RNA Synthesizer on a 15 micromole scale using cyanoethyl phosphoramidites and the above prepared CPG solid support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner to afford the desired oligonucleotide.

### EXAMPLE 61

| | |
|---|---|
| Synthesis of fully-modified 5′-d(GTT-CTC-GCT-GGT-GAG-TTT-C)-3′ | (SEQ ID NO: 2) |
| phosphorothioate 19-mer | |

Synthesis of above sequence is performed on an ABI 390Z DNA/RNA Synthesizer on a 15 micromole scale using cyanoethyl phosphoramidites and the above prepared CPG solid support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner to afford the desired phosphorothioate oligonucleotide.

### EXAMPLE 62

| | |
|---|---|
| Synthesis of fully-modified 5′-d(TCC-CGC-CTG-TGA-CAT-GCA-TT)-3′ | (SEQ ID NO: 1) |
| phosphorothioate 20-mer | |

Synthesis of above sequence is performed on a Pharmacia OligoPilot II Synthesizer on a 135 micromole scale using cyanoethyl phosphoramidites and the above prepared HL30 Primer support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner to afford the purified desired oligonucleotide.

### EXAMPLE 63

| | |
|---|---|
| Synthesis of fully-modified 5′-d(GTT-CTC-GCT-GGT-GAG-TTT-C)-3′ | (SEQ ID NO: 2) |
| phosphorothioate 19-mer | |

Synthesis of above sequence is performed on a Pharmacia OligoPilot II Synthesizer on a 323 micromole scale using cyanoethyl phosphoramidites and the above prepared Primer support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner to afford the phosphorothioate oligonucleotide.

### EXAMPLE 64

| | |
|---|---|
| Synthesis of fully-modified 5′-d(CTA-CGC-TTT-CCA-CGC-ACA-G)-3′ | (SEQ ID NO: 3) |
| phosphorothioate | |

Synthesis of above sequence is performed on a Pharmacia OligoPilot II Synthesizer on a 323 micromole scale using cyanoethyl phosphoramidites and the above prepared Primer support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner to afford the phosphorothioate oligonucleotide.

### EXAMPLE 65

| | |
|---|---|
| Synthesis of fully-modified 5′-d(TCC-CGC-CTG-TGA)-2′-methoxyethyl-(CAT-GCA- | (SEQ ID NO: 4) |
| TT)-3′ phosphorothioate 20-mer | |

Synthesis of above sequence is performed on an OligoPilot II DNA/RNA Synthesizer on a 282 μmole scale using cyanoethyl phosphoramidites and derivatized primar support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner.

### EXAMPLE 66

(SEQ ID NO: 4)

Synthesis of fully-modified 5′-d(TCC-CGC-CTG-
TGA)-2′-methoxyethyl-(CAT-GCA-TT)-3′
phosphorothioate 20-mer

Synthesis of above sequence is performed on a Pharmacia OligoPilot II Synthesizer at 250 μmole scale using cyanoethyl phosphoramidites and derivatized primar support. Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected and purified in the usual manner.

### EXAMPLE 67

Preparation of 7-Oxabicyclo-[2.2.1]hept-2-ene

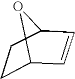

7-Oxabicyclo-[2.2.1]hept-2-ene is synthesized as per the literature procedure disclosed in Mirsadeghi et al., J. Org. Chem., 1985, 50, 4340-4345.

### EXAMPLE 68

Osmium Tetroxide Catalyst Solution

The content of a sealed vial of osmium tetroxide (1 g) is dissolved in 200 mL of purified t-butyl alcohol. The pale green solution is treated with 3-5 drops of 30% hydrogen peroxide and allowed to remain at room temperature for 1 day. If the solution becomes dark, the drop wise addition of 30% hydrogen peroxide is repeated until the pale green color persist. The resulting solution is stable for at least one year at room temperature. Each mL will contain 2×10⁻⁵ mole of osmium tetroxide.

### EXAMPLE 69

Dihydroxylation of Olefin to Give cis-Diol

7-Oxabicyclo-[2.2.1]hept-2-ene (0.934 mole) is taken in a 5 L three-necked flask fitted with a mechanical stirrer, reflux condenser with ice-water cooling and a heating mantle. Acetone (2500 mL) is added with stirring. A 30% hydrogen peroxide solution (500 mL) is added followed by osmium tetroxide solution (Example 2, 180 mL). Slow addition (1-2 hour) of osmium tetroxide solution is recommended. Gentle refluxing of reaction mixture with stirring is maintained for 7-8 h. The solid is washed with ether and dried in a vacuum oven at room temperature overnight. The acetone filtrate solution is concentrated to afford more of the product.

### EXAMPLE 70

Mono Protection of Diol with DMT Chloride

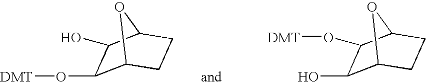

The cis-diol compound (Example 3, 0.1 mole) is taken in a 1 L round-bottomed flask and co-evaporated with anhydrous pyridine (200 mL). This step is repeated one more time to render the diol anhydrous. Pyridine (300 mL) is added and stirred using a magnetic stirrer at room temperature. Dimethoxytrityl chloride (FW 338.82; 1.2 equivalents) is slowly added as solid over a period of 3 hours. The solution is stirred overnight. All volatiles are removed under vacuum using rotavap. Toluene (200 mL) is added and rotavaped. This step is repeated one more time. The remaining crude material is purified by flash column chromatography on silica gel to afford the desired product.

### EXAMPLE 71

Succinylation of DMT Protected Compound

The DMT protected cis-diol compound (8.5 mmole) is dissolved in a mixture ethyl acetate (60 mL) and methylene chloride (6.6 mL). Triethyl amine (FW 101.19; 6 equivalent with respect to starting DMT compound) is added and stirred magnetically at room temperature. To this clear solution, succinic anhydride (FW 100.07; 4 equivalents with respect to starting DMT compound) is added as solid all at once. Stirring is continued overnight. The reaction mixture is diluted with ethyl acetate (100 mL) and washed with water (2×50 mL), brine (20 mL) and dried with magnesium sulfate. Filtration and concentration will give the desired product.

### EXAMPLE 72

Loading of DMT Protected Succinate to Controlled Pore Glass

Loading of the succinate molecule is performed similar to standard procedures for loading a nucleoside succinate using HBTU as activator and Hunig's base in acetonitrile as solvent. The unreacted sites are capped with acetic anhydride in pyridine in the presence of DMAP as catalyst. Loading is then checked using the standard UV method.

### EXAMPLE 73

Loading of DMT Protected Succinate to HL30 Amino-Derivatized Primer Support

Loading of the succinate molecule to amino-derivatized HL30 base bead is performed similar to standard procedures used to load a nucleoside succinate using HBTU as activator and Hunig's base in acetonitrile as solvent. The unreacted sites are capped with acetic anhydride in pyridine in presence of DMAP as catalyst. Loading is then checked using the standard UV method.

### EXAMPLE 74

Synthesis of Fully-Modified 5′-d(TCC-CGC-CTG-TGA-CAT-GCA-TT)-3′ Phosphorothioate 20-mer using DMT Protected Linker Molecule

Synthesis of above sequence is performed on an ABI 390Z DNA/RNA Synthesizer on a 15 micromole scale using cyanoethyl phosphoramidites and the above prepared CPG solid support (Example 72). Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected using ammonium hydroxide at 55° C. for 12 hours. The crude material is purified in the usual manner to afford the phosphorothioate oligonucleotide.

### EXAMPLE 75

Synthesis of fully-modified 5′-d(GCC-CAA-GCT-GGC-ATC-CGT-CA)-3′ phosphorothioate 20-mer using DMT protected linker molecule

Synthesis of above sequence is performed on an ABI 390Z DNA/RNA Synthesizer on a 15 micromole scale using cyanoethyl phosphoramidites and the above prepared CPG solid support (Example 72). Detritylation is performed using 3% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected using ammonium hydroxide at 55° C. for 12 hours. The crude material is purified in the usual manner to afford the phosphorothioate oligonucleotide.

### EXAMPLE 76

Synthesis of Fully-Modified 5′-d(TCC-CGC-CTG-TGA-CAT-GCA-TT)-3′ Phosphorothioate 20-mer using DMT Protected Linker Molecule

Synthesis of above sequence is performed on an Amersham Biosciences' Akta OligoPilot DNA/RNA Synthesizer on a 172 micromole scale using cyanoethyl phosphoramidites and the above prepared HL30 Primer solid support. Detritylation is performed using 10% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected using ammonium hydroxide at 55° C. for 12 hours. The crude material is purified in the usual manner to afford the phosphorothioate oligonucleotide.

### EXAMPLE 77

Synthesis of Fully-Modified 5′-d(GCC-CAA-GCT-GGC-ATC-CGT-CA)-3′ Phosphorothioate 20-mer using DMT Protected Linker Molecule

Synthesis of above sequence is performed on an Amersham Biosciences' Akta OligoPilot DNA/RNA Synthesizer on a 178 micromole scale using cyanoethyl phosphoramidites and the above prepared HL30 Primer solid support (Example 73). Detritylation is performed using 10% dichloroacetic acid in toluene (volume/volume). Sulfurization is performed using a 0.2 M solution of phenylacetyl disulfide in acetonitrile:3-picoline (1:1 v/v) for 2 minutes. At the end of synthesis, the support is washed with acetonitrile, cleaved, deprotected using ammonium hydroxide at 55° C. for 12 hours. The crude material is purified in the usual manner to afford the phosphorothioate oligonucleotide.

Those skilled in the art will appreciate that numerous changes and modifications may be made to the preferred embodiments of the invention and that such changes and modifications may be made without departing from the spirit of the invention. It is therefore intended that the appended claims cover all such equivalent variations as fall within the true spirit and scope of the invention.

What is claimed:

1. A compound of Formula I: 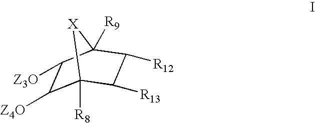
wherein:
X is CR₁₀R₁₁, O, S or NR₃;
R₃ is C₁-C₁₀ alkyl, substituted C₁-C₁₀ alkyl, —C(═O)alkyl, aryl or an amino protecting group;
each R₁₀ and R₁₁ is, independently, H, C₁-C₁₀ alkyl or substituted C₁-C₁₀ alkyl;
each R₁₂ and R₁₃ is, independently, H, C₁-C₁₀ alkyl, substituted C₁-C₁₀ alkyl, —C(═O)—R₄ or —C(═S)—R₄;
R₄ is —O—C₁-C₁₀ alkyl, —O—C₁-C₁₀ substituted alkyl, —O-aryl or —N(J₁)J₂;
J₁ is H or alkyl;
J₂ is alkyl or a nitrogen protecting group;
or J₁ and J₂ together with the nitrogen atom to which they are attached form a ring structure;
each R₈ and R₉ is, independently, H, C₁-C₁₀ alkyl or substituted C₁-C₁₀ alkyl;
each alkyl substituent is, independently, protected hydroxyl, alkoxy, benzyl, nitro, thioalkyl, aryl, thioaryl, thio substituted aryl, thioalkoxy, or halo;
one of Z₃ and Z₄ is a H or a hydroxyl protecting group and the other of Z₃ and Z₄ is a hydroxyl protecting group or -(L)ₙ-sm wherein when both Z₃ and Z₄ are hydroxyl protecting groups said protecting groups are orthogonal to each other; and
L is a linking moiety;
n is 0, or 1; and
sm is a support medium.

2. The compound of claim 1 wherein X is O, CH₂, S or NR₃;

3. The compound of claim 2 wherein X is O or CH₂.

4. The compound of claim 1 wherein X is O, one of Z₃ and Z₄ is -(L)ₙ-sm and the other of Z₃ and Z₄ is a hydroxyl protecting group or H.

5. The compound of claim 1 wherein X is NR₃ and R₃ is alkyl or —C(═O)alkyl.

6. The compound of claim 1 wherein R₈ and R₉ are both H.

7. The compound of claim 6 wherein L is succinyl, oxalyl, —C(═O)— or —C(═O)—NH—.

8. The compound of claim 1 wherein one of R₁₂ and R₁₃ is H and the other of R₁₂ and R₁₃ is C₁-C₁₀ alkyl or substituted C₁-C₁₀ alkyl.

9. The compound of claim 1 wherein R₁₂ and R₁₃ are each H.

10. The compound of claim 1 wherein one of Z₃ and Z₄ is -(L)ₙ-sm and the other of Z₃ and Z₄ is a hydroxyl protecting group.

11. The compound of claim 10 wherein said L is a succinyl or an oxalyl group.

12. The compound of claim 10 wherein said hydroxyl protecting group is dimethoxytrityl.

13. The compound of claim 1 wherein one of Z₃ and Z₄ is trimethylsilyl, triethylsilyl, t-butyldimethylsilyl, t-butyldiphenylsilyl, triphenylsilyl, benzoylformyl, acetyl, chloroacetyl, dichloroacetyl, trichloroacetyl, trifluoroacetyl, pivaloyl, benzoyl, p-phenylbenzoyl, 9-fluorenylmethoxycarbonyl, levulinyl or an acetoacetyl group and the other of of Z₃ and Z₄ is 4,4′-dimethoxytrityl, monomethoxytrityl, 9-phenylxanthen-9-yl, 9-(p-methoxyphenyl)xanthen-9-yl, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p,p-dinitrobenzhydryl, p-nitrobenzyl, triphenylmethyl, trimethylsilyl, triethylsilyl, t-butyldimethylsilyl, t-butyldiphenylsilyl, triphenylsilyl, benzoylformate, acetyl, chloroacetyl, trichloroacetyl, trifluoroacetyl, pivaloyl, benzoyl, p-phenylbenzoyl, mesyl, tosyl, 4,4′,4″-tris-(benzyloxy)trityl, 4,4′,4″-tris-(4,5-dichlorophthalimido)trityl, 4,4′,4″-tris(levulinyloxy)trityl, 3-(imidazolylmethyl)-4,4′-dimethoxytrityl, 4-decyloxytrityl, 4-hexadecyloxytrityl, 9-(4-octadecyloxyphenyl)xanthene-9-yl, 1,1-bis-(4-methoxyphenyl)-1′-pyrenyl methyl, p-phenylazophenyloxycarbonyl, 9-fluorenylmethoxycarbonyl, 2,4-dinitrophenylethoxycarbonyl, 4-(methylthiomethoxy)butyryl, 2-(methylthiomethoxymethyl)-benzoyl, 2-(isopropylthiomethoxymethyl)benzoyl, 2-(2,4-dinitrobenzenesulphenyloxymethyl)benzoyl, or a levulinyl group.

14. The compound of claim 1 wherein one of Z₃ and Z₄ has the formula: 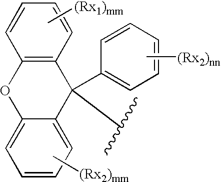
wherein
each Rx₁ is, independently, C₁-C₁₀ alkyl or branched C₁-C₁₀ alkyl;
each Rx₂ is, independently, C₁-C₁₀ alkyl or branched C₁-C₁₀ alkyl;
each Rx₃ is, independently, C₁-C₁₀ alkyl or branched C₁-C₁₀ alkyl;
each mm is, independently, 0, 1, 2 or 3; and
nn is 0, 1, 2 or 3.

15. The compound of claim 14 wherein nn is 0.

16. The compound of claim 14 wherein nn is 1 and Rx₃ is C₁-C₁₀ alkyl.

17. The compound of claim 14 wherein nn is 1 and Rx₃ is a methyl group in the para or ortho position of the phenyl ring.

18. The compound of claim 14 wherein the other of Z₃ and Z₄ is H.

19. The compound of claim 14 wherein one of Z₃ and Z₄ has one of the formulas:

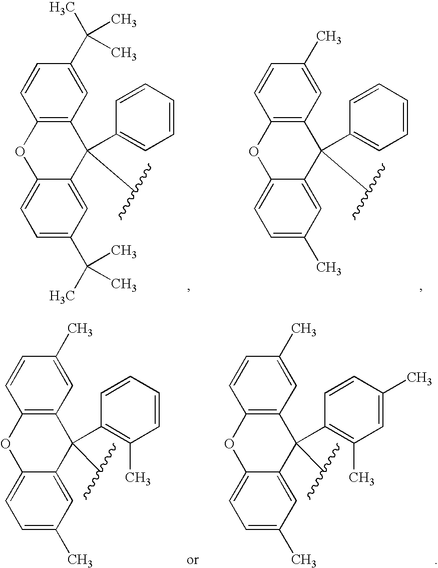

20. The compound of claim 19 wherein the other of Z₃ and Z₄ is H.

21. The compound of claim 1 having formula III: 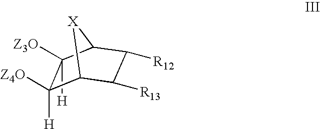
wherein:
X is O or CH₂;
each R₁₂ and R₁₃ is, independently, H, C₁-C₁₀ alkyl, substituted C₁-C₁₀ alkyl, —C(═O)—R₄ or —C(═S)—R₄;
R₄ is —O—C₁-C₁₀ alkyl, —O—C₁-C₁₀ substituted alkyl, —O-aryl or —N(J₁)J₂;
J₁ is H or alkyl;
J₂ is alkyl or a nitrogen protecting group;
or J₁ and J₂ together with the nitrogen atom to which they are attached form a ring structure;
one of Z₃ and Z₄ is a H or a hydroxyl protecting group and the other of Z₃ and Z₄ is a hydroxyl protecting group or -(L)ₙ-sm wherein when both Z₃ and Z₄ are hydroxyl protecting groups said protecting groups are orthogonal to each other;
L is a linking moiety;
n is 0 or 1; and
sm is a support medium.

22. The compound of claim 21 wherein one of Z₃ and Z₄ is -(L)ₙ-sm and the other of Z₃ and Z₄ a hydroxyl protecting group or H.

23. The compound of claim 22 wherein said L is a succinyl or an oxalyl group.

24. The compound of claim 22 wherein the other of Z₃ and Z₄ is dimethoxytrityl.

25. The compound of claim 21 wherein said support medium is a controlled pore glass, oxalyl-controlled pore glass, silica-containing particles, polymers of polystyrene, copolymers of polystyrene, copolymers of styrene and divinylbenzene, copolymers of dimethylacrylamide and N,N′-bisacryloylethylenediamine, soluble support medium, or PEPS.

26. The compound of claim 25, wherein said support medium is controlled pore glass, polymers of polystyrene or copolymers of polystyrene.

27. A method for functionalizing a support medium with a first monomeric subunit, comprising:
providing a support bound compound of Formula II: 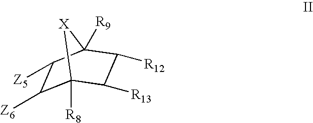
wherein:
X is CR₁₀R₁₁, O, S or NR₃;
R₃ is C₁-C₁₀ alkyl, substituted C₁-C₁₀ alkyl, —C(═O)alkyl, aryl or an amino protecting group;
each R₁₀ and R₁₁ is, independently, H, C₁-C₁₀ alkyl or substituted C₁-C₁₀ alkyl;
each R₁₂ and R₁₃ is, independently, H, C₁-C₁₀ alkyl, substituted C₁-C₁₀ alkyl, —C(═O)—R₄ or —C(═S)—R₄;
R₄ is —O—C₁-C₁₀ alkyl, —O—C₁-C₁₀ substituted alkyl, —O-aryl or —N(J₁)J₂;
J₁ is H or alkyl;
J₂ is alkyl or a nitrogen protecting group;
or J₁ and J₂ together with the nitrogen atom to which they are attached form a ring structure;
each R₈ and R₉ is, independently, H, C₁-C₁₀ alkyl or substituted C₁-C₁₀ alkyl;
each alkyl substituent is, independently, protected hydroxyl, alkoxy, benzyl, nitro, thioalkyl, aryl, thioaryl, thio substituted aryl, thioalkoxy, or halo;
one of Z₅ and Z₆ is a protected hydroxyl group and the other of Z₅ and Z₆ is —O-(L)ₙ-sm;
L is a linking moiety;
n is 0 or 1; and
sm is a support medium;
deprotecting said protected hydroxyl group to give a reactive hydroxyl group; and
treating said reactive hydroxyl group with a first monomeric subunit having an activated phosphorus group and a further protected hydroxyl group thereon for a time and under conditions sufficient to form a monomer-functionalized support medium.

28. The method of claim 27, further comprising:
treating said monomer-functionalized support medium with a capping agent; and
optionally, treating said monomer-functionalized support medium with an oxidizing agent.

29. The method of claim 28, further comprising:
deblocking said further protected hydroxyl group to give a reactive hydroxyl group;
treating said reactive hydroxyl group with a further monomeric subunit having an activated phosphorus group and a further protected hydroxyl group thereon for a time and under conditions sufficient to form an extended compound;
treating said extended compound with a capping agent;
optionally, treating said extended compound with an oxidizing or sulfurizing agent;
repeating the preceding four steps one or more times to form a further extended compound; and
treating said further extended compound with an oxidizing or sulfurizing agent to form an oligomeric compound.

30. The method of claim 29, wherein treating said further extended compound with said oxidizing agent to form said oligomeric compound removes protecting groups present on said oligomeric compound.

31. The method of claim 29, further comprising a step of treating said oligomeric compound with a reagent effective to cleave said oligomeric compound from said support medium.

32. The method of claim 31, wherein said reagent is a solution of ammonia.

33. The method of claim 31, wherein said cleaved oligomeric compound has a terminal hydroxyl group at the site of cleavage.

34. The method of claim 33, wherein said terminal hydroxyl group is attached to the 2′- or 3′-position of the nucleoside that is located at the 3′-terminus of said oligomeric compound.

35. The method of claim 34, wherein said terminal hydroxyl group is attached to the 2′-position of the nucleoside that is located at the 3′-termninus of said oligomeric compound.

36. The method of claim 29, wherein said treating of said reactive hydroxyl group with a further monomeric subunit is performed in the presence of an activating agent.

37. The method of claim 27, wherein X is O, S or NR₃.

38. The method of claim 27, wherein R₃ is alkyl or —C(═O)alkyl.

39. The compound of claim 27 wherein n is 1.

40. The method of claim 39, wherein L is succinyl, oxalyl, —C(═O)— or —C(═O)—NH—.

41. The method of claim 27, wherein said support medium is controlled pore glass, oxalyl-controlled pore glass, silica-containing particles, polymers of polystyrene, copolymers of polystyrene, copolymers of styrene and divinylbenzene, copolymers of dimethylacrylamide and N,N′-bisacryloylethylenediamine, soluble support medium or PEPS.

42. The method of claim 41, wherein said support medium is controlled pore glass, polymers of polystyrene or copolymers of polystyrene.

43. The method of claim 27, wherein one of Z₃ and Z₄ is trimethylsilyl, triethylsilyl, t-butyldimethylsilyl, t-butyldiphenylsilyl, triphenylsilyl, benzoylformyl, acetyl, chloroacetyl, dichloroacetyl, trichloroacetyl, trifluoroacetyl, pivaloyl, benzoyl, p-phenylbenzoyl, 9-fluorenylmethoxycarbonyl, levulinyl or acetoacetyl and the other of of Z₃ and Z₄ is 4,4′-dimethoxytrityl, monomethoxytrityl, 9-phenylxanthen-9-yl, 9-(p-methoxyphenyl) xanthen-9-yl, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p,p-dinitrobenzhydryl, p-nitrobenzyl, triphenylmethyl, trimethylsilyl, triethylsilyl, t-butyldimethylsilyl, t-butyldiphenylsilyl, triphenylsilyl, benzoylformate, acetyl, chloroacetyl, trichloroacetyl, trifluoroacetyl, pivaloyl, benzoyl, p-phenylbenzoyl, mesyl, tosyl, 4,4′,4″-tris-(benzyloxy)trityl, 4,4′,4″-tris-(4,5-dichlorophthalimido)trityl, 4,4′,4″-tris(levulinyloxy)trityl, 3-(imidazolylmethyl)-4,4′-dimethoxytrityl, 4-decyloxytrityl, 4-hexadecyloxytrityl, 9-(4-octadecyloxyphenyl)xanthene-9-yl, 1,1-bis-(4-methoxyphenyl)-1′-pyrenyl methyl, p-phenylazophenyloxycarbonyl, 9-fluorenylmethoxycarbonyl, 2,4-dinitrophenylethoxy carbonyl, 4-(methylthiomethoxy)butyryl, 2-(methylthiomethoxymethyl)-benzoyl, 2-(isopropylthiomethoxymethyl)benzoyl, 2-(2,4-dinitrobenzenesulphenyloxymethyl)benzoyl, or a levulinyl group.

44. The method of claim 27, wherein said monomeric subunit having an activated phosphorus group is a phosphoramidite, an H-phosphonate or a phosphate triester.

45. The method of claim 44, wherein said monomeric subunit is a phosphoramidite.

46. The method of claim 27, wherein Z₅ is an acid labile hydroxyl-protecting group.

47. The method of claim 29, wherein each of said further hydroxyl protecting groups is acid labile.

48. The method of claim 47, wherein said further hydroxyl protecting groups are removed by contact with an acid, wherein said acid is formic acid, acetic acid, chloroacetic acid, dichloroacetic acid, trichloroacetic acid, trifluoroacetic acid, benzenesulfonic acid, toluenesulfonic acid, or phenylphosphoric acid.

49. The method of claim 29, wherein said oligomeric compound is an oligonucleotide, modified oligonucleotide, oligonucleotide analog, oligonucleoside, oligonucleotide mimetic, hemimer, gapmer or chimera.

50. The method of claim 49, wherein said oligomeric compound is an oligonucleotide.

51. The method of claim 27, wherein one of Z₅ and Z₆ has the formula: 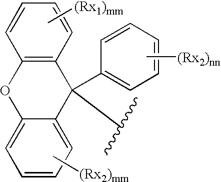
wherein
each Rx₁ is, independently, C₁-C₁₀ alkyl or branched C₁-C₁₀ alkyl;
each Rx₂ is, independently, C₁-C₁₀ alkyl or branched C₁-C₁₀ alkyl;
each Rx₃ is, independently, C₁-C₁₀ alkyl or branched C₁-C₁₀ alkyl;
each mm is, independently, 0, 1, 2 or 3; and
nn is 0, 1, 2 or 3.

52. The method of claim 51 wherein nn is 0.

53. The method of claim 51 wherein nn is 1 and Rx₃ is C₁-C₁₀ alkyl.

54. The method of claim 53 wherein Rx₃ is a methyl group in the para or ortho position of the phenyl ring.
